(19) United States
(12) Patent Application Publication
(10) Pub. No.: US 2024/0084240 A1
(43) Pub. Date: Mar. 14, 2024

(54) PREDICTION OF VIABILITY OF CELL CULTURE DURING A BIOMOLECULE MANUFACTURING PROCESS

(71) Applicant: GENENTECH, INC., South San Francisco, CA (US)

(72) Inventors: Arthi NARAYANAN, San Jose, CA (US); Aditya Avdhut WALVEKAR, Santa Clara, CA (US); Georo L. ZHOU, Richmond, CA (US); Nicholas RUMMEL, San Francisco, CA (US); Zheng LI, South San Francisco, CA (US); Steven J. MEIER, Burlingame, CA (US)

(73) Assignee: GENENTECH, INC., South San Francisco, CA (US)

(21) Appl. No.: 18/464,131

(22) Filed: Sep. 8, 2023

Related U.S. Application Data
(60) Provisional application No. 63/405,307, filed on Sep. 9, 2022.

(51) Int. Cl. C12M 1/36; G06N 3/08

(52) U.S. Cl. CPC C12M 41/48; G06N 3/08

(57) ABSTRACT

A method, system, and non-transitory computer readable medium for predicting cell viability of a cell culture in a bioreactor during a biomolecule manufacturing process are disclosed. In various embodiments, at least three manufacturing process parameters related to the process for manufacturing molecules are input into a machine learning model that is trained to predict cell viabilities. The trained machine learning model may then analyze the at least three manufacturing process parameters to generate an indicator of cell viability of the cell culture.

This application claims priority to U.S. Provisional Application Ser. No. 63/405,307, filed Sep. 9, 2022, which is incorporated by reference herein in its entirety.

### FIELD

In particular embodiments, this description is generally directed towards predicting viability of cells that produce molecules during a biomolecule manufacturing process. More specifically, this description provides methods and systems for training a machine learning model, and using the trained machine learning model, to predict an indicator of the viability of cells in cell culture.

### INTRODUCTION

The process for manufacturing biomolecules such as monoclonal antibodies includes a multitude of highly complex subprocesses such as, but not limited to, the culturing of cells, purification steps, in-process testing steps to evaluate and control the manufacturing process, and/or the like. An example of an in-process testing step is the sampling of the cell culture during the manufacturing process to evaluate the viability of the cells. Because such production steps can be time- and resource-intensive as well as being sources of product contamination, there is a need for techniques that allow determining the viability of cells during the manufacturing process without the need for product sampling.

### SUMMARY

In various embodiments, the disclosure provides systems, compositions, and methods for the analysis of biological samples. In some embodiments, the disclosure provides methods for evaluating a biological production process. In some embodiments, the disclosure provides methods for evaluating a culture medium. In some embodiments, the disclosure provides methods for evaluating a level of one or more culture components in a culture medium.

In aspects, computer-implemented methods, system, and media (CRM) are provided for predicting cell viability of a cell culture in a bioreactor during a biomolecule manufacturing process.

In one aspect, a computer-implemented method for predicting cell viability of a cell culture in a bioreactor during a biomolecule manufacturing process is described, the method comprising: receiving at least three manufacturing process parameters selected from one or both of a first set of manufacturing process parameters or a second set of manufacturing process parameters measured from the cell culture during the biomolecule manufacturing process; generating the indicator of cell viability of the cell culture by providing at least three manufacturing process parameters as input to a machine learning model that has been trained to generate an indicator of cell viability of the cell culture using training data comprising, for each of a plurality of cell cultures in a biomolecule manufacturing process, values of the at least three manufacturing process parameters and corresponding measured values of the indicator of cell viability, wherein: the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture.

In one aspect, the trained machine learning model has been trained with a manufacturing process training record including the one or both of the first set of manufacturing process parameters and the second set of manufacturing process parameters to generate the indicator of cell viability.

In an embodiment, any of the first set of manufacturing process parameters has an order of effect on the predicted indicator of cell viability of the cell culture that is higher than that of any of the second set of manufacturing process parameters.

In various instances, any one or more of the first set of manufacturing process parameters has an order of effect on the indicator of cell viability of the cell culture that is higher than that of any one or more of the second set of manufacturing process parameters. In various instances, the first set of process parameters includes time elapsed since an initiation of the biomolecule manufacturing process, total base added into the cell culture during the biomolecule manufacturing process, and a total volume of the cell culture in the bioreactor. Further, the second set of process parameters includes an amount of air sparged into the cell culture, an amount of dissolved oxygen in the cell culture, a pH of the cell culture, and a temperature of the cell culture.

In some embodiments of the aspects described above, the trained machine learning model is a neural network and in other embodiments, the trained machine learning model is a decision-tree based machine learning model. In certain embodiments, the trained machine learning model is a neural network, a decision tree, a random forest, a support vector machine, a Bayesian network, a regression tree, and/or the like. In a further embodiment for neural networks, the neural network is a deep neural network, a convolutional neural network (CNN), an artificial neural network (ANN), a recurrent neural network (RNN), a modular neural network (MNN), a residual neural network (RNN), an ordinary differential equations neural networks (neural-ODE), a squeeze and excitation embedded neural network, or a MobileNet. In a further embodiment, the ANN is a long short-term memory (LSTM) neural network. In another further embodiment, the regression tree is a gradient boosting machine (GBM) model (e.g., XGBoost). In some embodiments, the machine learning model is a probabilistic graphical model with a Bayesian network.

In an embodiment of any preceding aspect, the at least three manufacturing process parameters comprise one or more manufacturing process parameters that are measured by a sensor operationally connected to the bioreactor.

In other embodiments of any preceding aspect, the one or more manufacturing process parameters are selected from: one or more manufacturing process parameters are selected from: the pH of the cell culture, the temperature of the cell culture, the amount of dissolved oxygen in the cell culture, and a total volume of the cell culture, and/or the one or more manufacturing process parameters are each measured by a sensor selected from: a temperature probe, a dissolved oxygen probe, a pH probe, a Raman probe, a fluorescent probe, a liquid level imaging sensor, and a scale configured to weigh the cell culture, disposed within the bioreactor.

In some embodiments of any preceding aspect, the three manufacturing process parameters comprise one or more manufacturing process parameters that are obtained as an output of a controller operationally connected to the bioreactor.

In other embodiments of any preceding aspect, the one or more manufacturing process parameters are selected from: an amount of air sparged into the cell culture, an amount of carbon dioxide sparged into the cell culture, and an amount of oxygen sparged into the cell culture, and/or, the one or more manufacturing process parameters are each obtained as the output of an air flow controller configured to control flow of one or more of: the air sparged into the cell culture, the carbon dioxide sparged into the cell culture, and the oxygen sparged into the cell culture.

In various embodiments of the aspects described above, the first set of manufacturing process parameters further includes an amount of oxygen sparged into the cell culture; the first set of manufacturing process parameters listed in order of effect on the indicator of cell viability is shown in Table 1; and Table 1=

| Serial No. | Manufacturing process parameter |
|---|---|
| 1 | the time elapsed since the |
| | initiation of the biomolecule |
| | manufacturing process |
| 2 | the amount of oxygen sparged into |
| | the cell culture |
| 3 | the total volume of the cell culture |
| 4 | the total base added into the cell |
| | culture during the biomolecule |
| | manufacturing process |

In other embodiments of the aspects described above, the second set of manufacturing process parameters further includes an amount of carbon dioxide sparged into the cell culture; the second set of manufacturing process parameters listed in order of effect on the indicator of cell viability is shown in Table 2; and Table 2=

| Serial No. | Manufacturing process parameter |
|---|---|
| 1 | the amount of carbon dioxide |
| | sparged into the cell culture |
| 2 | the amount of dissolved oxygen in |
| | the cell culture |
| 3 | the pH of the cell culture |
| 4 | the amount of air sparged into the |
| | cell culture |
| 5 | the temperature of the cell culture |

In certain embodiments of the aspects described above, the first set of manufacturing process parameters listed in order of effect on the indicator of cell viability is shown in Table 3; and Table 3=

| Serial No. | Manufacturing process parameter |
|---|---|
| 1 | the time elapsed since the |
| | initiation of the biomolecule |
| | manufacturing process |
| 2 | the total base added into the cell |
| | culture during the biomolecule |
| | manufacturing process |
| 3 | the total volume of the cell culture |

In some embodiments of the aspects described above, the second set of manufacturing process parameters listed in order of effect on the indicator of cell viability is shown in Table 4; and Table 4=

| Serial No. | Manufacturing process parameter |
|---|---|
| 1 | the amount of oxygen sparged into |
| | the cell culture |
| 2 | the amount of carbon dioxide |
| | sparged into the cell culture |
| 3 | the temperature of the cell culture |
| 4 | the amount of air sparged into the |
| | cell culture |
| 5 | the pH of the cell culture |
| 6 | the amount of dissolved oxygen in |
| | the cell culture |

In various embodiments of the aspects described above, the second set of manufacturing process parameters listed in order of effect on the indicator of cell viability is shown in Table 5; and Table 5=

| Serial No. | Manufacturing process parameter |
|---|---|
| 1 | the amount of air sparged into the |
| | cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of carbon dioxide |
| | sparged into the cell culture |

In particular embodiments of the aspects described above, the second set of manufacturing process parameters listed in order of effect on the indicator of cell viability is shown in Table 6; and Table 6=

| Serial No. | Manufacturing process parameter |
|---|---|
| 1 | the pH of the cell culture |
| 2 | the amount of carbon dioxide |
| | sparged into the cell culture |
| 3 | the temperature of the cell culture |
| 4 | the amount of oxygen sparged into |
| | the cell culture |
| 5 | the amount of air sparged into the |
| | cell culture |
| 6 | the amount of dissolved oxygen in |
| | the cell culture |

In other embodiments of the aspects described above, the first set of manufacturing process parameters further includes an amount of carbon dioxide sparged into the cell culture; the first set of manufacturing process parameters listed in order of effect on the indicator of cell viability is shown in Table 7; and Table 7=

| Serial No. | Manufacturing process parameter |
|---|---|
| 1 | the time elapsed since the |
| | initiation of the biomolecule |
| | manufacturing process |
| 2 | the amount of carbon dioxide |
| | sparged into the cell culture |
| 3 | the total base added into the cell |
| | culture during the biomolecule |
| | manufacturing process |
| 4 | the total volume of the cell culture |

In other embodiments of the aspects described above, the second set of manufacturing process parameters further includes an amount of oxygen sparged into the cell culture; the second set of manufacturing process parameters listed in order of effect on the indicator of cell viability is shown in Table 8; and Table 8=

| Serial No. | Manufacturing process parameter |
|---|---|
| 1 | the amount of air sparged into the |
| | cell culture |
| 2 | the amount of dissolved oxygen in |
| | the cell culture |
| 3 | the temperature of the cell culture |
| 4 | the pH of the cell culture |
| 5 | the amount of oxygen sparged into |
| | the cell culture |

In some embodiments of the aspects described above, the second set of manufacturing process parameters further includes an amount of oxygen sparged into the cell culture; the second set of manufacturing process parameters listed in order of effect on the indicator of cell viability is shown in Table 9; and Table 9=

| Serial No. | Manufacturing process parameter |
|---|---|
| 1 | the temperature of the cell culture |
| 2 | the amount of oxygen sparged into |
| 3 | the pH of the cell culture |
| 4 | the amount of air sparged into the |
| | cell culture |
| 5 | the cell culture the amount of |
| | dissolved oxygen in the cell |
| | culture |

In other embodiments of the aspects described above, the first set of manufacturing process parameters listed in order of effect on the indicator of cell viability is shown in Table 10; and Table 10=

| Serial No. | Manufacturing process parameter |
|---|---|
| 1 | the time elapsed since the |
| | initiation of the biomolecule |
| | manufacturing process |
| 2 | the total volume of the cell culture |
| 3 | the total base added into the cell |
| | culture during the biomolecule |
| | manufacturing process |

In particular embodiments of the aspects described above, the second set of manufacturing process parameters listed in order of effect on the indicator of cell viability is shown in Table 11; and Table 11=

| Serial No. | Manufacturing process parameter |
|---|---|
| 1 | the amount of carbon dioxide |
| | sparged into the cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of air sparged into the |
| | cell culture |

In some embodiments, the methods also comprises receiving at least three manufacturing process parameters selected from one or both of a first set of manufacturing process parameters, or a second set of manufacturing process parameters measured from the cell culture during the biomolecule manufacturing process. Further, the method comprises analyzing the at least three manufacturing process parameters using a trained machine learning model to generate an indicator of cell viability of the cell culture.

In an embodiment, the at least three manufacturing process parameters are selected from: time elapsed since an initiation of the biomolecule manufacturing process, total base added into the cell culture during the biomolecule manufacturing process, a total volume of the cell culture in the bioreactor, amount of air sparged into the cell culture, an amount of dissolved oxygen in the cell culture, a pH of the cell culture, and a temperature of the cell culture.

In various embodiments of any of the preceding aspects, the biomolecule manufacturing process is a process for manufacturing a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid and/or wherein the cells in the cell culture, produce a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid.

The methods or system of any of the present aspects may have any one or more of the features described in relation to the first aspect.

The methods of the present aspect may comprise predicting cell viability of a cell culture in a bioreactor during a biomolecule manufacturing process, according to any embodiment of the first aspect.

In certain methods of any preceding aspect, the trained machine learning model comprises parameters that assign higher importance to the manufacturing process parameters selected from the first set than to the manufacturing process parameters selected from the second set, optionally wherein the trained machine learning model comprises parameters that assign respective importance to each of the manufacturing process parameters, wherein: (i) the at least three manufacturing process parameters are selected from or include all of the parameters of Tables 1 and 2, and the order of importance of the manufacturing process parameters are provided in Tables 1 and 2; (ii) the at least three manufacturing process parameters are selected from or include all of the parameters of Tables 3 and 4, and the order of importance of the manufacturing process parameters are provided in Tables 3 and 4; (iii) the at least three manufacturing process parameters are selected from or include all of the parameters of Tables 5 and 6, and the order of importance of the manufacturing process parameters are provided in Tables 5 and 6; (iv) the at least three manufacturing process parameters are selected from or include all of the parameters of Tables 7 and 8, and the order of importance of the manufacturing process parameters are provided in Tables 7 and 8; (v) the at least three manufacturing process parameters are selected from or include all of the parameters of Tables 7 and 9, and the order of importance of the manufacturing process parameters are provided in Tables 7 and 9; (vi) the at least three manufacturing process parameters are selected from or include all of the parameters of Tables 10 and 11, and the order of importance of the manufacturing process parameters are provided in Tables 10 and 11. In a further aspect, there is provided a computer-implemented method of obtaining a tool for predicting cell viability of a cell culture in a bioreactor during a biomolecule manufacturing process, the method comprising: receiving training data comprising, for each of a plurality of cell cultures in a biomolecule manufacturing process: values of at least three manufacturing process parameters selected from one or both of a first set of manufacturing process parameters or a second set of manufacturing process parameters measured from the cell culture during the biomolecule manufacturing process; and corresponding measured values of the indicator of cell viability; and training a machine learning model to generate an indicator of cell viability of the cell culture using said training data, the machine learning model taking as input the values of the at least three manufacturing process parameters and providing as output a predicted indicator of cell viability; wherein any of the first set of manufacturing process parameters has an order of effect on the predicted indicator of cell viability of the cell culture that is higher than that of any of the second set of manufacturing process parameters; the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture. In certain embodiments, one or more manufacturing process parameters are selected from: the pH of the cell culture, the temperature of the cell culture, the amount of dissolved oxygen in the cell culture, and a total volume of the cell culture, and/or the one or more manufacturing process parameters are each measured by a sensor selected from: a temperature probe, a dissolved oxygen probe, a pH probe, a Raman probe, a fluorescent probe, a liquid level imaging sensor, and a scale configured to weigh the cell culture, disposed within the bioreactor.

Also described herein according to a further aspect is a system comprising a non-transitory memory storing instructions; and a processor coupled to the non-transitory memory and configured to read the instructions from the non-transitory memory to cause the system to perform any of the methods of the above aspect.

Also described herein according to a further aspect is a non-transitory computer-readable medium (CRM) having stored thereon computer-readable instructions executable that when executed by a processor cause the processor to implement the method of any embodiment of a method of obtaining a tool for predicting cell viability of a cell culture as described herein.

The method of an aspect may further comprise identifying a control action based on the predicted indicator of cell viability. The control action may be selected from: stopping the cell culture, measuring product critical quality attributes (CQA) like glycosylation etc., measuring the product titer, harvesting the spent media, changing the value of one or more manufacturing process parameters (for e.g. pH shift, temp shift, adding base, sparging of CO₂ or other gas, etc.), starting or continuing a feed, adding fresh cell culture media and/or cell culture supplements, sending an alert to a computing device or user interface, etc.

Also described herein are methods of monitoring and/or controlling a biomolecule manufacturing process comprising a cell culture in a bioreactor, the methods comprising predicting cell viability of the cell culture using a method according to any embodiment of the first aspect. The method may further comprise comparing the predicted cell viability to one or more reference value. The method may further comprise determining whether the process is operating normally based on the comparison. The method may further comprise issuing an alert when it is determined that the process is not operating normally. The reference value may be an expected cell viability or range of cell viabilities, for example obtained from previous processes that are known to have operated normally. A process may be considered to have operated normally if the product of the process met one or more predetermined critical quality attributes (CQAs). The reference value may be a previously predicted cell viability for the same process. For example, the comparison may determine whether the cell viability has decreased or decreased by more than a predetermined value, compared to a prediction at a previous time point of the process. An alert may be issued when the comparison indicates that the cell viability has decreased or decreased by more than a predetermined value, compared to a prediction at a previous time point of the process.

In certain embodiments of the aspects described above, the at least three manufacturing process parameters do not include any manufacturing process parameter that is obtained by analyzing cells sampled from the cell culture.

In other embodiments of the aspects described above, the at least three manufacturing process parameters do not include any manufacturing process parameter that is measured by analyzing a sample of cell culture obtained from the bioreactor.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a cell viability prediction system, in accordance with various embodiments.

FIG. 2 is a workflow of a process for training a machine learning model to predict viability of cells producing molecules during a molecule manufacturing process, in accordance with various embodiments.

FIG. 3 is a flowchart of a machine learning model-based process for generating an indicator of cell viability of a cell culture in a bioreactor during a biomolecule manufacturing process, in accordance with various embodiments.

FIGS. 4A-4C show a bar graph of feature importance scores on the x-axis vs. various molecule manufacturing process parameters on the y-axis (FIG. 4A), a heatmap (FIG. 4B), and a table (FIG. 4C) illustrating the identification of biomolecule manufacturing process parameters for use in training a machine learning model to predict viability of cells, in accordance with various embodiments.

FIGS. 5A-5B show a graph (FIG. 5A) and a table (FIG. 5B) illustrating the training of a machine learning model trained to predict viability of cells producing biomolecules during a biomolecule manufacturing process, in accordance with various embodiments. FIG. 5A provides a graph of measured vs. predicted viability of cells.

FIG. 5B: provides a table with a listing of performance evaluation metrics, for e.g., R2, mean, residuals, RMSE, etc.

FIGS. 6A-6B show a graph (FIG. 6A) and a table (FIG. 6B) illustrating the testing of a machine learning model to predict viability of cells producing biomolecules during a biomolecule manufacturing process, in accordance with various embodiments.

FIGS. 7A-7C show graphs (FIGS. 7A and 7B) and a table (FIG. 7C) illustrating the testing of a machine learning model trained to predict viability of cells producing biomolecules during a biomolecule manufacturing process, in accordance with various embodiments.

FIGS. 8A-8F show graphs illustrating viability predictions of a machine learning model for multiple types of biomolecules produced by a process for manufacturing the biomolecules, in accordance with various embodiments.

FIGS. 9A-9B show bar graphs illustrating the viability distribution for multiple types of biomolecules (FIG. 9A) and per biomolecule type (FIG. 9B), in accordance with various embodiments.

FIG. 10 is a block diagram of a computer system in accordance with various embodiments.

FIG. 11 illustrates an example neural network that can be used to implement a deep learning neural network in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Mammalian and other cell lines are frequently used in the production of desired biological molecules (“biomolecules”). Cell culture operating parameter optimization helps achieve high expression of product with acceptable product quality profiles. These parameters are physical, chemical and biological in nature. Physical parameters include, but are not limited to, temperature, gas flow rate and agitation speed; Chemical parameters include, but are not limited to, dissolved oxygen and carbon dioxide, pH, osmolality, redox potential and metabolite levels, including substrate, amino acid and waste by-products; Biological parameters are used for determining the physiological state of the culture and include, but are not limited to, viable cell concentration (VCC), cell viability, a variety of intracellular and extra-cellular measurements such as NADH, LDH levels, mitochondrial activity, cell cycle analysis, etc. to name a few. Variations in the micro-environment parameters from optimal levels can have a dramatic impact on culture performance, productivity and product quality. A typical stirred tank bioreactor is equipped with temperature, pressure, agitation, pH and dissolved oxygen sensors and/or controls. Cell culture operating strategies and parameters that effect cell culture environmental conditions, include but are not limited to, dissolved oxygen (DO), pH, osmolality, dissolved CO₂, mixing, hydrodynamic shear, etc. The cell culture environment consequently influences process performance which can be measured by parameters such as cell growth, metabolite concentrations, product titer and product quality. These cell culture manufacturing processes are well-described in Li et al., Cell culture processes for monoclonal antibody production, mAbs 2:5, 466-477; September/October 2010; Landes Bioscience; the contents of which are hereby incorporated by reference.

Optimization of biomolecule manufacturing processes means measuring, monitoring and adjusting for example, the cell's health, titer, product quality attributes in the bioreactor. Real-time and near real-time monitoring of cell culture manufacturing processes are part of the process analytical technology (PAT) paradigms for upstream bioprocessing. The responses measured can enable rapid feedback to perturbations that can otherwise lead to batch failures. Historically, real-time monitoring of bioreactor processes monitored parameters such as pH, dissolved oxygen, and temperature, or analytical results such as cell growth and metabolites through manual daily sampling. In order to reduce sample error and to increase throughput, real-time and near real-time instruments have been developed. These and recent advances (including dielectric spectroscopy, NIR, off-gas spectrometry, integrated at-line HPLC, and nanofluidic devices for monitoring cell growth and health, metabolites, titer, etc.) are discussed in Current Opinion in Biotechnology, Volume 71, October 2021, Pages 191-197.

However, the use of machine learning (ML) models in cell culture manufacturing processes is still in its infancy. So far, Raman probes have been utilized in ML models. No ML models have been developed to monitor or to predict cell viability so far. Various embodiments encompassed herein provide methods and systems that utilize training of a machine learning model for predicting an indicator of cell viability of biological cells that produce certain biomolecules (alternatively referred herein as “molecules”) and that are being manufactured in a cell-based system. In various embodiments, such a strategy for measuring and predicting cell viability improves on present methods by allowing one to circumvent costly, time-consuming methods for testing the cells during the process. They also provide for streamlining analysis processes that otherwise would be subject to disadvantages such as in-process sampling and testing steps fraught with the potential for contamination, for example. However, in some embodiments, in-process sampling and testing steps may also be utilized.

In various embodiments, the cell viability in the present disclosure is an example of a measure of the output of the desired biological molecules. The disclosure encompasses embodiments for production of any type of biomolecule, including proteins (biopharmaceuticals, cytokines, fusion proteins, growth factors, monoclonal antibodies, complex antibodies, antibody fragments, virus or viral particles, vaccines, etc.); lipids; carbohydrates; and nucleic acids. The cells in the cell culture produce the monoclonal antibody, complex antibody, antibody fragment, virus or viral particles, vaccines, etc.

Therefore, the present disclosure encompasses a complex yet efficacious system and related methods in which decision-making for one or more parameters can occur (prior to and/or) in real-time. In various embodiments, the viability of the cells is related to the manufacturing output, and based on the trained machine learning model one can modify, manipulate, or toggle one or more parameters to improve the output. In particular embodiments, this may occur in the absence of testing, thereby maintaining the integrity of the cell-based system and saving cost and labor and risk of contamination. The present system and processes allow for manipulation of choice of parameter, order of parameter inputs, magnitude of parameter content, a combination thereof, etc., as a result of predictive information from the model. In particular embodiments, such manipulations occur at the process conditional level instead of at the cellular level, as with known cell-based manufacturing systems, and allow for prompt modification(s) dependent upon one or more outputs, for example.

In various embodiments, the methods and systems concern predicting the viability of cells that produce immunotherapeutic molecules including antibodies, for example monoclonal antibodies for detection, diagnostic, and/or therapeutic purposes. In various embodiments, the methods and systems concern predicting the viability of cells that produce antibodies of any kind, including monoclonal antibodies, based on a process for manufacturing the biomolecules in a certain environment. In specific embodiments, the methods predict the viability of cells producing monoclonal antibodies based on a process of manufacturing antibodies in a cell culture in a bioreactor. The cell culture environment allows analysis of antibody-producing cells in the presence of one or more manipulable process parameters, and values for such parameters are received by a trained machine learning model that provides an informative output(s) that facilitates determination of the viability of the antibody-producing cells. In specific embodiments, the encompassed methods train a machine learning model based on the viability of the antibody-producing cells that may be applied to subsequent cells. In particular embodiments, information learned from the model may be applied to cells produced subsequently in the same culture and/or applied to cells produced in a parallel culture (that may or may not have the same conditions at the time of testing).

### II. Definitions

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “about” refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or ±1% as understood by a person of skill in the art.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

The term “antibody” as used herein refers to any immunologic binding agent such as IgG, IgM, IgA, IgD and IgE and also refers to any antibody-like molecule that has an antigen binding region, and includes antibody fragments such as Fab′, Fab, F(ab′)₂, single domain antibodies (DABs), Fv, scFv (single chain Fv), and the like. The antibody may be monoclonal or humanized, in specific embodiments.

The term “bioreactor” as used herein refers to an apparatus suitable for housing and growing a cell culture, including on a manufacturing scale in some embodiments.

The term “cell culture” as used herein refers to growth of cells in an artificial environment under suitable conditions.

The term “molecule” as used herein refers to substances that are produced by cells and may include carbohydrates, lipids, nucleic acids, and proteins. The terms “molecule” and “biomolecule” may be used interchangeably. In embodiments, molecule or biomolecule refers to a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid.

The term “indicator of cell viability” as used herein refers to a measure of cell viability indicating the level of viability of cell culture that produce the biomolecules. For example, an indicator can be a percent, so an “indicator of cell viability” can be “percent viability,” which may be expressed by (the number of live cells/the number of dead cells)*100. Or the “indicator of cell viability” can be from readouts such as VCD, PCV (see below), or from assays such as cell viability (including but not limited to dye exclusion, calorimetric, Fluorescence-live cell assays, etc.), cell proliferation, cell toxicity, cell imaging, or metabolic assays such as ATP-based assay, etc. The cells may be any kind of cell and may be tailored to suitability for production of the desired molecule. The cells may be eukaryotic or prokaryotic. The cells may be single-celled organisms, animal cells, or plant cells, as examples.

The term “VCD” as used herein refers to viable cell density (#x 10\hat{ }6 of viable cells/mL of culture).

The term “PCV” as used herein refers to packed cell volume (volume of cells/total volume)*100.

As used herein, an “artificial neural network” or “neural network” (NN) may refer to mathematical algorithms or computational models. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may receive training data (learning examples) and learn how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

### III. Prediction of Viability of Cells that Produce Molecules During a Molecule Manufacturing Process

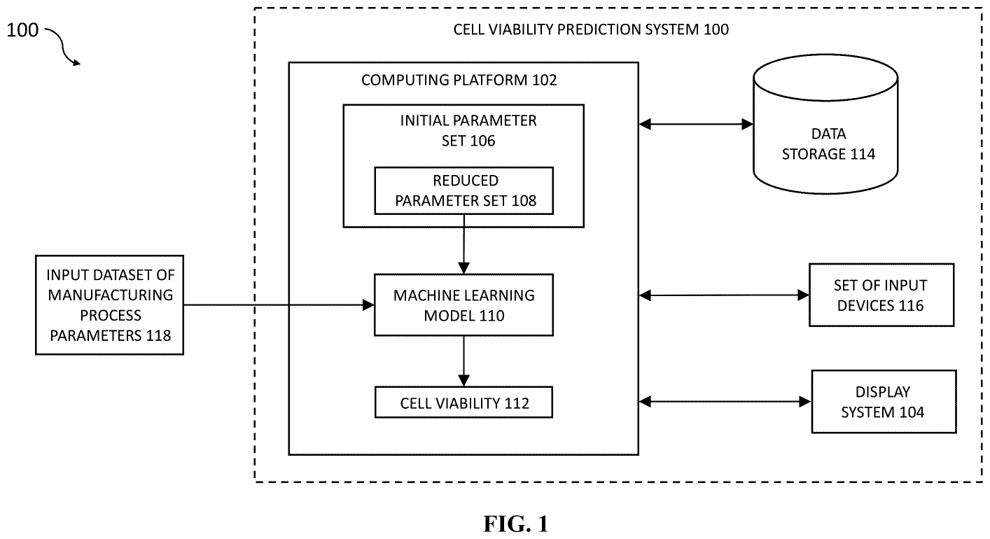

FIG. 1 is a block diagram of a cell viability prediction system 100 in accordance with various embodiments. Cell viability prediction system 100 uses a machine learning model 110 to predict an indicator (e.g., percent) of viability of cells that produce molecules during a process for manufacturing the molecules in a cell culture in a bioreactor. In some instances, the manufacturing process can be a molecule manufacturing batch process or continuous process (e.g., perfusion process). In specific embodiments, the process is noninvasive. In various embodiments, the process allows for continuous or periodic monitoring and adjustment to maintain optimum conditions within the bioreactor. In specific embodiments, the process allows for maintaining optimum nutrient and waste levels in the culture, including within predefined acceptable ranges. In particular aspects there is monitoring of any stage of the process in real time. In particular embodiments, the system can use an open loop or closed loop control for monitoring one or more manufacturing process parameters and then automatically change or vary one or more components, such as, e.g., flow of a parameter component in or out of the bioreactor. In some embodiments, the change may include flow in of a first parameter component and flow out of a second or subsequent parameter component that may or may not occur substantially at the same time. Any manufacturing process parameter may comprise, in certain embodiments, any fluid, compound, molecule, or substance that can increase the mass of the manufactured molecule in the cell culture.

Examples of molecules that are produced by the batch or continuous process include but are not limited to therapeutic antibodies (e.g., monoclonal antibodies (mAbs)). Cell viability prediction system 100 includes computing platform 102, data storage 114, set of input devices 116, and display system 104.

Computing platform 102 may take various forms. In various embodiments, computing platform 102 includes a single computer (or computer system) or multiple computers in communication with each other. In other examples, computing platform 102 takes the form of a cloud computing platform. In various embodiments, computing platform 102 may be communicatively coupled with data storage 114, set of input devices 116, display system 104, or a combination thereof. In various embodiments, data storage 114, set of input devices 116, display system 104, or a combination thereof may be considered part of or otherwise integrated with computing platform 102. Thus, in some examples, computing platform 102, data storage 114, set of input devices 116, and display system 104 may be separate components in communication with each other, but in other examples, some combination of these components may be integrated together.

In various embodiments, the initial parameter set 106 may include manufacturing process parameters that are related to the process of manufacturing molecules in a cell culture in a bioreactor and can be measured during the manufacturing process and/or otherwise obtained from a database (e.g., from data storage 114) coupled to the computing platform 102. For example, molecules such as, but not limited to, mAbs can be produced in a bioreactor, and parameters related to the manufacturing process of such molecules in the bioreactor may be measured or otherwise obtained (e.g., and stored in data storage 114). Non-limiting examples of said manufacturing process parameters include, e.g., amount of carbon dioxide sparged into the cell culture in which the molecules are produced, amount of air sparged into the cell culture, amount of oxygen sparged into the cell culture, pH primary, amount or concentration of dissolved oxygen (dO₂) in the cell culture, total base added into the cell culture during the molecule manufacturing process (“base total”), agitation control output, back pressure, total volume of the cell culture, pH output, temperature of the cell culture, the temperature of the bioreactor/vessel shell (“jacket temperature”), temperature output of a bioreactor/vessel shell controller, culture duration or time elapsed since the initiation of the molecule manufacturing process, glucose concentration, lactate concentration, sodium concentration, ammonium concentration, osmolality, packed cell volume (PCV), carbon dioxide concentration, oxygen concentration, and/or the like, related to the manufacturing process of molecules.

As used herein, the CO₂ sparge refers to the introduction of CO₂ bubbled through the cell culture. In specific embodiments, CO₂ is sparged in as a pH control mechanism to maintain pH at a set point. Base may be added to the cell culture to increase pH, while CO₂ and/or acid may be added to decrease pH. Since even a small deviation of 0.1 units from the optimal pH value can significantly impact culture growth and metabolism, in particular glucose consumption and lactate production, pH is an important variable to measure and control. Cell culture media usually contains sodium bicarbonate as buffering agent, and pH is usually tightly controlled with a combination of CO₂ sparging to reduce pH and base addition to increase it. High pH (>7.0) is usually preferred for initial cell growth phase, which is usually accompanied by lactate accumulation. When lactate accumulation exceeds the buffering capacity of the culture medium, pH drifts downward, which could trigger base addition leading to increased osmolality of the culture medium. This could be risky in cell lines that synthesize excessive amounts of lactate since high pH, high lactate and high osmolality cascade often causes delayed cell growth and accelerated cell death. When cell growth has ceased, lactate is either produced at a much lower rate or consumed. The concomitant upward drift in pH is counteracted by CO₂ sparging. Thus, the pH set-point and control strategy, e.g., dead band, are intimately linked to dissolved CO₂ levels, base consumption for pH control and therefore, osmolality (from Li et al., mAbs 2:5, 466-477; September/October 2010; Landes Bioscience; the contents of which are hereby incorporated by reference).

As used herein, the O₂ sparge refers to the introduction of O₂ bubbled through the cell culture to support cell respiration and growth. Dissolved oxygen is typically controlled at a specific set point, usually between 20-50% of air saturation in order to prevent dissolved oxygen limitation, which might lead to excessive lactate synthesis, and excessively high dissolved oxygen concentrations that could lead to cytotoxicity.

As used herein, the air sparge refers to the introduction of air bubbled through the cell culture.

In various embodiments, the pH of the cell culture in the bioreactor may be in a range of from about 6.5 to about 7.5 (also refer to CO₂ sparging above and pH control). In some embodiments, the pH of the cell culture may be in a range from about 6.5 to about 7.4, about 6.5 to about 7.3, about 6.5 to about 7.2, about 6.5 to about 7.1, about 6.5 to about 7.0, about 6.5 to about 6.9, about 6.5 to about 6.8, about 6.5 to about 6.7, about 6.5 to about 6.6, about 6.6 to about 7.5, about 6.6 to about 7.4, about 6.6 to about 7.3, about 6.6 to about 7.2, about 6.6 to about 7.1, about 6.6 to about 7.0, about 6.6 to about 6.9, about 6.6 to about 6.8, about 6.6 to about 6.7, about 6.7 to about 7.5, about 6.7 to about 7.4, about 6.7 to about 7.3, about 6.7 to about 7.2, about 6.7 to about 7.1, about 6.7 to about 7.0, about 6.7 to about 6.9, about 6.7 to about 6.8, about 6.8 to about 7.5, about 6.8 to about 7.4, about 6.8 to about 7.3, about 6.8 to about 7.2, about 6.8 to about 7.1, about 6.8 to about 7.0, about 6.8 to about 6.9, about 6.9 to about 7.5, about 6.9 to about 7.4, about 6.9 to about 7.3, about 6.9 to about 7.2, about 6.9 to about 7.1, about 6.9 to about 7.0, about 7.0 to about 7.5, about 7.0 to about 7.4, about 7.0 to about 7.3, about 7.0 to about 7.2, about 7.0 to about 7.1, about 7.1 to about 7.5, about 7.1 to about 7.4, about 7.1 to about 7.3, about 7.1 to about 7.2, about 7.2 to about 7.5, about 7.2 to about 7.4, about 7.2 to about 7.3, about 7.3 to about 7.5, about 7.3 to about 7.4, or about 7.4 to about 7.5. In specific embodiments, the pH of the cell culture may be at least, or no more than, about 6.5, about 6.6, about 6.7, about 6.8, about 6.9, about 7.0, about 7.1, about 7.2, about 7.3, about 7.4, or about 7.5. The term “pH primary” as used herein may refer to the pH of the cell culture that is measured by a pH sensor onboard the bioreactor (e.g., measuring “online” in real-time). The term “pH output” as used herein may refer to pH values measured by a pH controller or sensor (e.g., and may be used to determine whether base or CO₂ should be adjusted to increase or decrease, the pH of the cell culture).

In certain embodiments, the amount or concentration of dissolved oxygen (dO₂) in the bioreactor may be in a range of from about 0% to about 100%. Dissolved oxygen is typically controlled at a specific set point, usually between 20-50% of air saturation. The dO₂ may be in a range of 0% to about 80%, 0% to about 75%, 0% to about 60%, 0% to about 50%, 0% to about 40%, 0% to about 20%, 0% to about 10%, !% to about 5%, about 5% to about 100%, about 5% to about 80%, about 5% to about 75%, about 5% to about 60%, about 5% to about 50%, about 5% to about 40%, about 5% to about 20%, about 5% to about 10%, about 10% to about 100%, about 10% to about 80%, about 10% to about 75%, about 10% to about 60%, about 10% to about 50%, about 10% to about 40%, about 10% to about 20%, about 20% to about 100%, about 20% to about 80%, about 20% to about 75%, about 20% to about 60%, about 20% to about 50%, about 20% to about 40%, about 40% to about 100%, about 40% to about 80%, about 40% to about 75%, about 40% to about 60%, about 40% to about 50%, about 50% to about 80%, about 50% to about 75%, about 50% to about 60%, about 60% to about 100%, about 60% to about 80%, about 60% to about 75%, about 75% to about 100%, about 75% to about 80%, or about 80% to about 100%. In some embodiments, the dO₂ may be at least, or no more than, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, the temperature of the cell culture may be in a range of from about 30° C. to about 37° C. In some embodiments, the temperature may be in the range of about 30° C. to about 36° C., about 30° C. to about 35° C., about 30° C. to about 34° C., about 30° C. to about 33° C., about 30° C. to about 32° C., about 30° C. to about 31° C., about 31° C. to about 37° C., about 31° C. to about 36° C., about 31° C. to about 35° C., about 31° C. to about 34° C., about 31° C. to about 33° C., about 31° C. to about 32° C., about 32° C. to about 37° C., about 32° C. to about 36° C., about 32° C. to about 35° C., about 32° C. to about 34° C., about 32° C. to about 33° C., about 33° C. to about 37° C., about 33° C. to about 36° C., about 33° C. to about 35° C., about 33° C. to about 34° C., about 34° C. to about 37° C., about 34° C. to about 36° C., about 34° C. to about 35° C., about 35° C. to about 37° C., about 35° C. to about 36° C., or about 36° C. to about 37° C. In some embodiments, the temperature may be at least, or no more than, about 30° C., about 31° C., about 32° C., about 33° C., about 34° C., about 35° C., about 36° C., or about 37° C. In certain embodiments, the culture duration may be from about 3 days to about 30 days, from about 5 days to about 25 days, from about 5 days to about 20 days, from about 6 days to about 16 days, from about 8 days to about 12 days, from about 10 days to about 12 days, including values and subranges therebetween. In some embodiments, the culture duration may be from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days. In some embodiments, the culture duration may be at least, or no more than, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days.

In various embodiments, the amount of glucose in the media of the culture may be in a range from about 0 g/L to about 50 g/L, from about 0 g/L to about 40 g/L, from about 0 g/L to about 30 g/L, from about 0 g/L to about 20 g/L, from about 0 g/L to about 10 g/L, from about 10 g/L to about 50 g/L, from about 10 g/L to about 40 g/L, from about 10 g/L to about 30 g/L, from about 10 g/L to about 20 g/L, from about 20 g/L to about 50 g/L, from about 20 g/L to about 40 g/L, from about 20 g/L to about 30 g/L, from about 30 g/L to about 50 g/L, from about 30 g/L to about 40 g/L, or from about 40 g/L to about 50 g/L, including values and subranges therebetween. In various embodiments, the amount of glucose in the media of the culture may be at least, or no more than, about 0 g/L, about 10 g/L, about 20 g/L, about 30 g/L, about 40 g/L, or about 50 g/L.

In various embodiments, the amount of lactate produced by cells in the media may be an indirect measure for cell growth of the culture and may be in a range from about 0 g/L to about 50 g/L, from about 0 g/L to about 40 g/L, from about 0 g/L to about 30 g/L, from about 0 g/L to about 20 g/L, from about 0 g/L to about 10 g/L, from about 10 g/L to about 50 g/L, from about 10 g/L to about 40 g/L, from about 10 g/L to about 30 g/L, from about 10 g/L to about 20 g/L, from about 20 g/L to about 50 g/L, from about 20 g/L to about 40 g/L, from about 20 g/L to about 30 g/L, from about 30 g/L to about 50 g/L, from about 30 g/L to about 40 g/L, or from about 40 g/L to about 50 g/L, including values and subranges therebetween. In various embodiments, the amount of lactate in the media of the culture may be at least, or no more than, about 0 g/L, about 10 g/L, about 20 g/L, about 30 g/L, about 40 g/L, or about 50 g/L. In some embodiments, lactate is a waste product in cell culture, and so the amount of lactate is monitored to ensure that the level of lactate in the culture is no more than a particular concentration, such as no more than about 0 g/L, about 5 g/L, about 10 g/L, about 15 g/L, about 20 g/L, about 25 g/L, about 30 g/L, about 35 g/L, about 40 g/L, about 45 g/L, or about 50 g/L.

In certain embodiments, the amount of sodium in the media may be in a range of from about 0 to about 250 mM, about 0 to about 200 mM, about 0 to about 150 mM, about 0 to about 100 mM, about 0 to about 50 mM, about 0 to about 25 mM, about 25 mM to about 250 mM, about 25 mM to about 200 mM, about 25 mM to about 150 mM, about 25 mM to about 100 mM, about 25 mM to about 50 mM, about 50 mM to about 250 mM, about 50 mM to about 200 mM, about 50 mM to about 150 mM, about 50 mM to about 100 mM, about 100 mM to about 250 mM, about 100 mM to about 200 mM, or about 200 mM to about 250 mM, including values and subranges therebetween. In some embodiments, the amount of sodium in the media may be at least, or no more than, 5 mM, 10 mM, 15 mM, 20 mM, 25 mM, 50 mM, 75 mM, 100 mM, 125 mM, 150 mM, 175 mM, 200 mM, 225 mM, or 250 mM.

In certain embodiments, the amount of ammonium in the media may be in a range of from about 0 to about 50 mM, including values and subranges therebetween. In certain embodiments, the amount of ammonium in the media may be in a range of from about 0 to about 40 mM, about 0 to about 30 mM, about 0 to about 20 mM, about 0 to about 10 mM, about 10 mM to about 50 mM, about 10 mM to about 40 mM, about 10 mM to about 30 mM, about 10 mM to about 20 mM, about 20 mM to about 50 mM, about 20 mM to about 40 mM, about 20 mM to about 30 mM, about 30 mM to about 50 mM, about 30 mM to about 40 mM, or about 40 mM to about 50 mM. In some embodiments, the amount of ammonium in the media may be at least, or no more than, 0, 5 mM, 10 mM, 15 mM, 20 mM, 25 mM, 30 mM, 35 mM, 40 mM, 45 mM, or 50 mM.

In various embodiments, the osmolality of the culture may be in a range of from about 0 to about 700 mOsm, including values and subranges therebetween. In some embodiments, the osmolality of the culture may be in a range of from about 0 mOsm to about 700 mOsm, from about 0 mOsm to about 600 mOsm, from about 0 mOsm to about 500 mOsm, from about 0 mOsm to about 400 mOsm, from about 0 mOsm to about 300 mOsm, from about 0 mOsm to about 200 mOsm, from about 0 mOsm to about 100 mOsm, from about 0 mOsm to about 50 mOsm, from about 50 mOsm to about 700 mOsm, from about 50 mOsm to about 600 mOsm, from about 50 mOsm to about 500 mOsm, from about 50 mOsm to about 400 mOsm, from about 50 mOsm to about 300 mOsm, from about 50 mOsm to about 200 mOsm, from about 50 mOsm to about 100 mOsm, from about 100 mOsm to about 700 mOsm, from about 100 mOsm to about 600 mOsm, from about 100 mOsm to about 500 mOsm, from about 100 mOsm to about 400 mOsm, from about 100 mOsm to about 300 mOsm, from about 100 mOsm to about 200 mOsm, from about 200 mOsm to about 700 mOsm, from about 200 mOsm to about 600 mOsm, from about 200 mOsm to about 500 mOsm, from about 200 mOsm to about 400 mOsm, from about 200 mOsm to about 300 mOsm, from about 300 mOsm to about 700 mOsm, from about 300 mOsm to about 700 mOsm, from about 300 mOsm to about 600 mOsm, from about 300 mOsm to about 500 mOsm, from about 300 mOsm to about 400 mOsm, from about 400 mOsm to about 700 mOsm, from about 400 mOsm to about 600 mOsm, from about 400 mOsm to about 500 mOsm, from about 500 mOsm to about 700 mOsm, or from about 600 mOsm to about 700 mOsm, In specific embodiments, the osmolality of the culture may be at least, or no more than, 0 mOsm, 25 mOsm, 50 mOsm, 75 mOsm, 100 mOsm, 150 mOsm, 200 mOsm, 250 mOsm, 250 mOsm, 300 mOsm, 350 mOsm, 400 mOsm, 450 mOsm, 500 mOsm, 550 mOsm, 600 mOsm, 650 mOsm, or 700 mOsm. In various embodiments, the carbon dioxide of the culture may be in a range of from about 0 to about 250 mmHg, including values and subranges therebetween. In some embodiments, the carbon dioxide of the culture may be in a range of from about 0 to about 250 mmHg, about 0 to about 200 mmHg, about 0 to about 150 mmHg, about 0 to about 100 mmHg, about 0 to about 50 mmHg, about 50 mmHg to about 250 mmHg, about 50 mmHg to about 200 mmHg, about 50 mmHg to about 150 mmHg, about 50 mmHg to about 100 mmHg, about 100 mmHg to about 250 mmHg, about 100 mmHg to about 200 mmHg, about 100 mmHg to about 150 mmHg, about 150 mmHg to about 250 mmHg, about 150 mmHg to about 200 mmHg, or about 150 mmHg to about 250 mmHg. In some embodiments, the carbon dioxide of the culture may be at least, or no more than 1, 5 mmHg, 10 mmHg, 15 mmHg, 20 mmHg, 25 mmHg, 30 mmHg, 35 mmHg, 40 mmHg, 45 mmHg, 50 mmHg, 75 mmHg, 100 mmHg, 125 mmHg, 150 mmHg, 175 mmHg, 200 mmHg, 225 mmHg, or 250 mmHg. In various embodiments, the oxygen (O₂) of the culture may be in a range of from about 0 to about 250 mmHg, including values and subranges therebetween. In some embodiments, the O₂ of the culture may be in a range of from about 0 to about 250 mmHg, about 0 to about 200 mmHg, about 0 to about 150 mmHg, about 0 to about 100 mmHg, about 0 to about 50 mmHg, about 50 mmHg to about 250 mmHg, about 50 mmHg to about 200 mmHg, about 50 mmHg to about 150 mmHg, about 50 mmHg to about 100 mmHg, about 100 mmHg to about 250 mmHg, about 100 mmHg to about 200 mmHg, about 100 mmHg to about 150 mmHg, about 150 mmHg to about 250 mmHg, about 150 mmHg to about 200 mmHg, or about 150 mmHg to about 250 mmHg. In some embodiments, the O₂ of the culture may be at least, or no more than 1, 5 mmHg, 10 mmHg, 15 mmHg, 20 mmHg, 25 mmHg, 30 mmHg, 35 mmHg, 40 mmHg, 45 mmHg, 50 mmHg, 75 mmHg, 100 mmHg, 125 mmHg, 150 mmHg, 175 mmHg, 200 mmHg, 225 mmHg, or 250 mmHg.

In various embodiments, the range of viable cell density (VCD) is from about 0 to about 10′ viable cells/mL of culture, including values and subranges therebetween. In some embodiments, the range of VCD is at least 10², 10¹, 10⁴, 10⁵, 10⁶, or 10⁷ or more viable cells/mL.

In various embodiments, PCV is in the range of from about 0% to about 25%, from about 0% to about 20%, from about 0% to about 15%, from about 0% to about 10%, from about 10% to about 25%, from about 10% to about 20%, from about 10% to about 15%, 15% to about 25%, 15% to about 20%, or 20% to about 25%, including values and subranges therebetween. In some embodiments, the PCV is at least, or no more than, about 1%, about 5%, about 10%, about 15%, about 20%, or about 25%.

In some embodiments, the total volume of the cell culture comprises a volume suitable to allow proliferation of cells to a desired concentration in the culture. Examples of volumes include at least, or no more than, 100 mL to 500 mL, 100 mL to 1 L, 100 mL to 2 L, 100 mL to 5 L, 100 mL to 10 L, 100 mL to 20 L, 1 L to 3 L, 1 L to 5 L, 1 L to 10 L, or 1 L to 20 L. In some cases, the volume is volume of 20 L or less, 10 L or less, 5 L or less, 4 L or less, 3 L or 15 less, 2 L or less, 1 L or less, or 0.1 L or less. In some embodiments, the volume of the cell culture is at least, or no more than, 500 L, at least 1000 L, at least 2000 L, at least 3000 L, at least 4000 L, at least 5000 L, at least 7500 L, at least 10000 L, at least 12500 L, at least 15000 L, at least 20000 L, at least 100000 L, or more. In some embodiments, the manufacturing-scale bioreactor culture has a working volume of 2000 L or 15000 L. In some embodiments, a manufacturing-scale bioreactor culture has a working volume in a range of 500 L to 1000 L, 500 L to 2500 L, 500 L to 5000 L, 500 L to 10000 L, 500 L to 15000 L, 500 L to 20000 L, 500 L to 100000 L, 2000 L to 5000 L, 2000 L to 10000 L, 2000 L to 15000 L, 2000 L to 20000 L, 2000 L to 100000 L, 15000 L to 20000 L, 15000 L to 100000 L, 20000 L to 50000 L, 20000 L to 100000 L, or 50000 L to 100000 L.

In various embodiments, the initial parameter set 106 may be obtained from online and/or offline measurements associated with the process of manufacturing molecules in the bioreactor. That is, the manufacturing process parameters of the initial parameter set 106 may be obtained via online and/or offline measurements. Online measurements can be measurements performed by sensors and/or controllers with outputs that are operationally connected to the bioreactor. Offline measurements can be measurements that are performed using standalone instruments on samples that are extracted from the bioreactor (e.g., manually).

For example, online measurements may be performed by one or more probes that are operationally coupled to the bioreactor, or cell culture therein, and configured to measure manufacturing process parameters associated with the bioreactor and/or the cell culture. For example, a scale may be operationally coupled to the bioreactor and may be configured to measure the weight or volume of the cell culture. As another example, a temperature probe, a dissolved oxygen probe, or a pH probe may be operationally coupled to the bioreactor and/or the cell culture to measure the temperature of the cell culture, the amount or concentration of dissolved oxygen in the cell culture, or the pH of the cell culture (“pH primary”), respectively. In some instances, the temperature probe may also be configured to measure the temperatures of other components or process features of the manufacturing process (e.g., in addition to or besides the temperature of the cell culture). For example, the temperature probe may be configured to measure the temperature of the bioreactor/vessel shell (“jacket temperature”).

In some instances, one or more online measurements may be obtained from controllers configured to control one or more manufacturing process parameters. For example, a controller that is operationally coupled to the bioreactor and configured to control a manufacturing process parameter may have an output associated with the manufacturing process parameter. In such cases, the manufacturing process parameter may be measured by reading the output of the controller.

In some embodiments, the controller can be in communication and control thermocirculators, load cells, control pumps, and receive information from various sensors and probes. For instance, the controller may control and/or monitor the pH, the oxygen tension, dissolved carbon dioxide, the temperature, the agitation conditions, the alkali condition, the pressure, foam levels, and the like. For example, based on pH readings from a pH probe, the controller may be configured to regulate pH levels by adding requisite amounts of acid or alkali. The controller may also use a carbon dioxide gas supply to decrease pH. Similarly, the controller can receive temperature information and control fluids being feed to a water jacket surrounding the bioreactor for increasing or decreasing temperature.

In various embodiments, the bioreactor may be operationally coupled to an air flow controller configured to control the flow of air, carbon dioxide, oxygen, a combination thereof, etc., sparged into the cell culture, and the output of the controller may be read to measure or determine the amount of air, carbon dioxide, oxygen, etc., respectively, that is sparged into the cell culture. As another example, a temperature controller may be configured to control the temperature of the bioreactor vessel, and the output of the temperature controller may be read to measure the jacket temperature and/or the temperature of the cell culture. In some instances, a pH controller may be configured to control the pH of the cell culture, and the reading of this pH controller (referred herein as “pH output”) may be used as a measure of the pH of the cell culture. In some cases, the pH output measurement may be used to regulate the pH of the cell culture. For example, based on the pH output measurement, a base may be added, or CO₂ sparged (and/or acid added), into the cell culture, to increase or decrease the pH of the cell culture, respectively.

In certain cases, the measure (e.g., amount or concentration) of one or more offline parameters, such as glucose, lactate, sodium, ammonium, a combination thereof, etc., may be obtained by respective commercial kits (e.g., Cedex analyzer from Roche). Although buffers are included in the bioreactor to control acidity of the culture media, production of metabolic and other acids by the cells may upset the equilibrium of the culture and require adjustment of one or more parameters (e.g., the pH) by standard methods. In certain aspects, the base total may be measured based on the rate that base is added into the cell culture and the duration of the addition (e.g., the product of the rate and the duration gives the base total). In certain aspects, the osmolality may be measured using an offline freezing point osmometer. In one example, a controller is operationally coupled to the bioreactor and configured to control the intensity of agitation as agitation control output. In some cases, a controller that regulates back pressure is utilized and may comprise a normally-closed valve configured to provide an obstruction or a pressure hold to flow, thereby regulating the upstream (back) pressure. “Back” in this context means against the natural flow of fluid and refers to upstream pressure that is held, or maintained, in a variety of production vessels to provide the right conditions for separation and processing. As one example, one or more gas sensors may be operationally coupled to the bioreactor and configured to sense and regulate the amount of gas in the bioreactor, including, for example, for carbon dioxide and/or oxygen. The PCV in the bioreactor may be measured using commercially available tubes and equipment for the determination of biomass (such as a micro capillary centrifuge). Or, cell density m-ay can be measured using cell counter sensor or an optical density sensor.

In various embodiments, the manufacturing process parameters of the initial parameter set 106 may constitute a machine learning model training records or dataset that may be used to train the machine learning model 110 to predict an output of the molecule manufacturing process. An example of the output can be cell viability 112 of the cells that produce the molecules during the molecule manufacturing process. For instance, as discussed above, the initial parameter set 106 may include a variety of manufacturing process parameters that are measured (e.g., via sensors, probes, controllers, combinations thereof, etc.) or otherwise obtained during the molecule manufacturing process. In such cases, the viability of the cells that produce the molecules may also be measured or otherwise obtained (e.g., by measuring the cell viability of a manual sample of the cell culture). In such cases, the training records or dataset for training the machine learning model 110 can be prepared by pairing one or more manufacturing process parameters with the measured cell viabilities. The training of the machine learning model then comprises inputting this training dataset (e.g., which includes the manufacturing process parameters of the initial parameter set 106 and the cell viability associated therewith) to the machine learning model 110 to predict the associated cell viability.

In some instances, it may be desirable to remove one or more of the manufacturing process parameters in the training dataset, such as to reduce the size of the training dataset. For example, the one or more of the manufacturing process parameters to be removed may have low importance for or impact on the training of the machine learning model 110. In another example, a manufacturing process parameters may not have unique contribution to the training of the machine learning model 110. For instance, two manufacturing process parameters may be highly correlated (e.g., and as such may have at least substantially similar contribution to the training of the machine learning model 110). In such cases, one of the two manufacturing process parameters may not have unique contribution to the training of the machine learning model 110 and may be redundant. In such cases, it may be desirable to remove from the training dataset those redundant and/or low-importance manufacturing process parameters, such as to save computing resources and time as well as reduce the complexity of the training of the machine learning model 110.

In various embodiments, the reduced parameter set 108 may be obtained from the initial parameter set 106 by pre-processing the latter using a machine learning feature selection technique to remove one or more of the manufacturing process parameters (e.g., those that are redundant, those that have low impact on the training of the machine learning model 110, a combination thereof, and/or the like). In some instances, the term “features” refers to the manufacturing process parameters in the training dataset, and examples of the feature selection technique include filter methods, wrapper methods, or combination thereof. A reduced training dataset may then be generated based on the reduced parameter set 108 and the cell viability associated with the manufacturing process parameters of the reduced parameter set 108.

Filter methods include statistics techniques that measure the importance of features in a dataset for training a machine learning model. As such, the application of filter methods to the initial parameter set 106 includes the application of the statistical techniques to measure the importance of the manufacturing process parameters in the initial parameter set 106. In such cases, the manufacturing process parameters identified as being of low importance based on the statistical techniques may be removed from the initial parameter set 106 to generate the reduced parameter set 108. The reduced training dataset may then include the reduced parameter set 108 and the cell viability associated with the manufacturing process parameters of the reduced parameter set 108.

The statistical techniques in the filter methods include the computations of information gain/mutual information, Fisher score, correlation coefficient, variance threshold, and/or the like. Information gain or mutual information refers to the amount of information provided by a manufacturing process parameter (e.g., in the initial parameter set 106) for identifying the target variable, i.e., the viability of the cells that produce the molecules. Information gain or mutual information measures the dependency of the manufacturing process parameter over the viability of the cells. An information gain that is zero indicates that the manufacturing process parameter and the cell viability are independent variables (i.e., no information about the cell viability can be obtained from the manufacturing process parameter). Manufacturing process parameters with information gain less than a threshold amount may be removed from the initial parameter set 106 to generate the reduced parameter set 108 (e.g., and consequently the reduced training dataset for training the machine learning model 110).

In some instances, Fisher scores of the manufacturing process parameters in the initial parameter set 106 can be computed to evaluate the individual importance of the manufacturing process parameters to the training of the machine learning model 110. A larger or smaller Fisher score associated with a manufacturing process parameter indicates that the manufacturing process parameter has higher or lower importance, and in such cases, manufacturing process parameters of the initial parameter set 106 with Fisher score no greater than a threshold Fisher score may be removed from the initial parameter set 106 to generate the reduced parameter set 108 (e.g., and consequently the reduced training dataset for training the machine learning model 110).

Filter methods can utilize correlation coefficients to measure linear relationships or correlations between any two manufacturing process parameters in the initial parameter set 106. Larger or smaller correlation coefficients between pairs of manufacturing process parameters indicate higher or lower, respectively, correlations between the pairs of manufacturing process parameters. Because highly correlated manufacturing process parameters contribute or provide largely similar information towards the training of the machine learning model 110, larger correlation coefficients may be understood to indicate parameter redundancies in the initial parameter set 106. In some instances, when a pair of manufacturing process parameters have associated therewith a correlation coefficient exceeding a threshold correlation coefficient, one of the pair may be removed from the initial parameter set 106 to generate the reduced parameter set 108 (e.g., and consequently the reduced training dataset for training the machine learning model 110).

In some instances, filter methods include computations configured to evaluate the variability of the initial parameter set 106. Because manufacturing process parameters that have low variance are expected to contribute little towards the training of the machine learning model 110 to predict cell viability 112, manufacturing process parameters with associated variance less than a variance threshold may be removed from the initial parameter set 106 to generate the reduced parameter set 108 (e.g., and consequently the reduced training dataset for training the machine learning model 110). In some instances, the variance of a manufacturing process parameter X may be computed using the expression Var [X]=E[(X−E[X])²], where E(X) is the expected value of the manufacturing process parameter X.

In various embodiments, wrapper methods of selecting manufacturing process parameters from the initial parameter set 106 to form the reduced parameter set 108 utilize algorithms that evaluate the performance of the machine learning model 110 for different subsets of the initial parameter set 106. In some instances, different subsets of the manufacturing process parameters of the initial parameter set 106 may be formed and these subsets may be provided to the machine learning model 110 as inputs for predicting the cell viability 112. The performance of the subsets may be evaluated by comparing the predicted cell viability 112 with the measured cell viability 112 associated with the initial parameter set 106. When the predicted cell viability 112 substantially matches the measured cell viability 112, the subset of manufacturing process parameters that resulted in the predicted cell viability 112 may be viewed as an optimal set of manufacturing process parameters to use in training the machine learning model 110. In such cases, the reduced parameter set 108 may be the same as or may be formed based on that subset of manufacturing process parameters.

The machine learning model 110 can be but is not limited to a neural network, a decision tree, a random forest, a support vector machine, a Bayesian network, a regression tree, and/or the like. The neural network can be a deep neural network, a convolutional neural network (CNN), an artificial neural network (ANN), a recurrent neural network (RNN), a modular neural network (MNN), a residual neural network (RNN), an ordinary differential equations neural networks (neural-ODE), a squeeze and excitation embedded neural network, a MobileNet, etc. The ANN can be a long short-term memory (LSTM) neural network. The regression tree can be a gradient boosting machine (GBM) model (e.g., XGBoost). In some instances, the machine learning model 110 can be a probabilistic graphical model with a Bayesian network.

In various embodiments, as discussed above, the machine learning model 110 can be trained with the reduced parameter set 108 to predict an indicator of cell viability 112. After training, the trained machine learning model 110 can be used to predict the indicator of viability of cells that produce molecules during a process for manufacturing the molecules in a cell culture in a bioreactor, provided an input dataset 118 of manufacturing process parameters that are associated with that process for manufacturing the molecules is provided to the machine learning model 110. For example, one can measure or otherwise obtain the manufacturing process parameters of the initial parameter set 106 using sensors, probes, controllers, a combination thereof, etc., operationally coupled to the bioreactor, and these measurements can be compiled as the input dataset 118 of manufacturing process parameters. In some instances, this input dataset 118 can be pre-processed as discussed above to generate a reduced input dataset from which redundant, low-important, etc., manufacturing process parameters are removed. This reduced input or the input dataset 118 may then be provided or input into the trained machine learning model 110 to predict the viability 112 of cells that produce the molecules during the molecule manufacturing process.

In various embodiments, the predicted indicator of cell viability 112 can be a percentage, a ratio, etc., that can indicate the viability of the cells that produce the molecules during the manufacturing process. That is, as used herein, the term “indicator of cell viability” may refer to measures of cell viability that indicate the level of the viability of the cell culture that produce the molecules. For example, the indicator of cell viability can be percent viability, which may correspond to the percentage of cells that are alive compared to the total number of cells (e.g., computed as number of alive cells*100/(number of alive cells+number of dead cells)). In some instances, the indicator of cell viability can be a ratio that may be scaled differently as desired (e.g., expressed in a scale of 1 to 10, versus 1 to 100 in the case of percentages). Further, in some instances, the indicator of cell viability 112 can be defined in terms of the ratio of the number of alive cells to the number of dead cells (e.g., and again scaled as desired). Generally, it is to be understood that cell viability can be defined in any manner that captures the relationship between the number of alive cells to the number of dead cells, and as such indicates the viability of the cells that produce the molecules during the molecule manufacturing process. For instance, the indicator of cell viability can be the following ratio, (number of alive cells−number of dead cells)/(number of alive cells+number of dead cells), scaled as desired (e.g., multiplied by hundred for percentages, etc.). In the instant disclosure, the prediction of cell viability may be understood to refer to the prediction or generation of an indicator of cell viability.

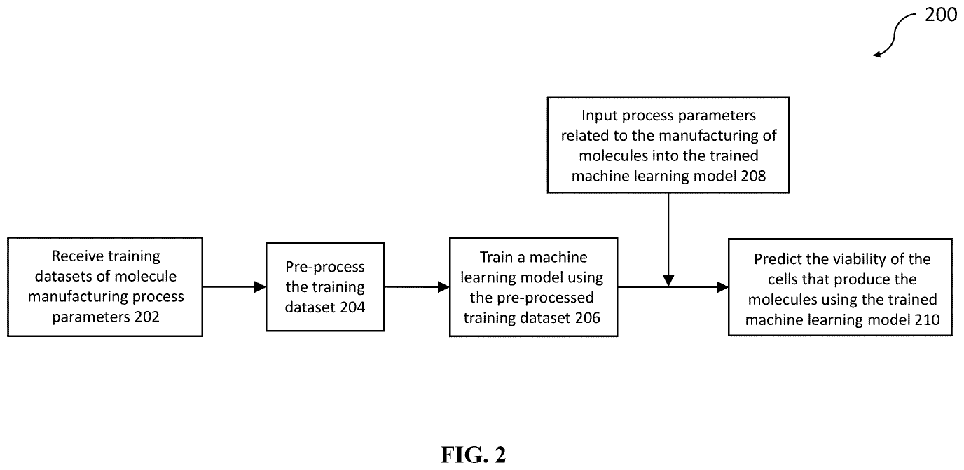

FIG. 2 is a workflow of a process 200 for training a machine learning model to predict viability of cell culture that produce molecules during a molecule manufacturing process, in accordance with various embodiments. In various embodiments, process 200 is implemented using a system, such as, the cell viability prediction system 100 of FIG. 1 to predict an indicator of viability of cells that produce molecules during a molecule manufacturing process. For example, a technician may be tasked with producing first molecules in a cell culture in a bioreactor. In such cases, the technician may wish to determine the viability of the cells that produce the first molecules without necessarily having to sample the cell culture to avoid contamination, and/or minimize delays and cost, etc. The technician may then utilize process 200 to train a machine learning model to predict cell viabilities and use the trained machine learning model to predict an indicator of cell viability of the cells that produce the first molecules in the bioreactor. In particular embodiments, the indicator of cell viability of the cells is an indicator of the amount of molecules produced in the bioreactor.

It should be appreciated that a manufacturing process parameter may refer to any component of a culture, including but not limited to serum components, nutrient components, waste components, biological cells, biological products, culture parameter(s) (that may be a molecular parameter, a cellular parameter, or a chemical parameter). In some embodiments, a culture parameter comprises any physicochemical or cellular characteristic of the culture including at least a nutrient, protein, peptide, amino acid, carbohydrate, growth factors, trace elements (e.g., cobalt, nickel, etc.), cytokine, salt, metal salt, fatty acids, lipids (e.g., cholesterol, steroids, and mixtures thereof), vitamins (group B vitamins, such as B12, vitamin A, vitamin E, riboflavin, thiamine, biotin, and mixtures thereof), the level of one or more constituents, the tonicity of a culture, the osmolality of a culture, the pH of a culture, the level of a cell in a culture, the total volume of the culture, and other similar parameters. When a nutrient is a parameter, the nutrient may comprise proteins, fats, carbohydrates (sugars, dietary fiber), vitamins, minerals, iron, sodium, mixtures thereof, and so forth. When a protein is a parameter, the protein may comprise antibodies, contractile proteins, enzymes, hormonal proteins, structural proteins, storage proteins, and/or transport proteins. When a carbohydrate is a parameter, the carbohydrate may include complex sugars and/or simple sugars, and may include glucose, maltose, fructose, galactose, and mixtures thereof. When an amino acid is a parameter, the amino acid may be glycine, alanine, valine, leucine, isoleucine, methionine, proline, phenylalanine, tryptophan, serine, threonine, asparagine, glutamine, tyrosine, cysteine, lysine, arginine, histidine, aspartic acid, glutamic acid, mixtures thereof, single stereoisomers thereof, racemic mixtures thereof and may also include non-standard amino acids, e.g., 4-hydroxyproline, ε-N,N,N-trimethyllysine, 3-methylhistidine, 5-hydroxylysine, O-phosphoserine, y-carboxyglutamate, γ-N-acetyllysine, ω-N-methylarginine, N-acety !serine, N,N,N-trimethyalanine, N-formy !methionine, γ-aminobutyric acid, histamine, dopamine, thyroxine, citrulline, ornithine, b-cyanoalanine, homocysteine, azaserine, and S-adenosylmethionine.

In particular embodiments upon producing cell cultures, one aspect of the methods includes lowering product variability and increasing product quality by increasing control of the manufacturing process parameter(s) within the cell culture. One or more components of the process including the media may be maintained within predefined ranges by manipulating the manufacturing process parameter(s). In specific embodiments, one can systematically manipulate one or more process variables, such as cell culture media components and/or culture conditions, and observe the impact or ultimate outcome in the propagating cell culture. In specific embodiments, the minimum and maximum of a predefined range of a given manufacturing process parameter may be within a certain percentage of each other, such as within at least, or no more than, 0.05, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% or more of each other.

At step 202, a machine learning model training dataset of manufacturing process parameters may be received, for example at the cell viability prediction system 100, for use in training a machine learning model to predict viability of the cells that produce the first biomolecules in the bioreactor. In some instances, the training dataset may include records of manufacturing process parameters associated with the manufacturing of second biomolecules and the viabilities of the cells that produce the second molecules. For example, the records may be measurements of manufacturing process parameters and cell viabilities taken during prior manufacturing processes conducted to produce the second biomolecules. In some instances, the second molecules may be the same as or different from the first biomolecules. In some instances, the training dataset may include records of one or more second biomolecules. In such cases, at least one of the one or more second biomolecules may be the same as the first biomolecules, or none of the one or more biomolecules may be the same as the first biomolecules. For instance, the first molecules that the technician is manufacturing may be a first monoclonal antibody. In such case, the training dataset may include records of manufacturing process parameters measured or otherwise obtained during the manufacturing of one or more second monoclonal antibodies and the cell viabilities associated with these one or more second monoclonal antibodies. In some cases, the first monoclonal antibody may be the same as at least one of the one or more second monoclonal antibodies. In other cases, the first monoclonal antibody may be different from each of the one or more second monoclonal antibodies. In some embodiments, a first monoclonal antibody recognizes a first antigen, and a second monoclonal antibody recognizes a second antigen that is different from the first. In certain embodiments, a first monoclonal antibody recognizes a first antigen and a second monoclonal antibody also recognizes the first antigen, but the first and second monoclonal antibodies are different (e.g., have one or more different complementarity-determining regions (CDRs)).

### EXAMPLES

In various embodiments, the records of the training dataset may include the manufacturing process parameters (e.g., of the initial parameter set 106 of FIG. 1) measured for the manufacturing process of the second molecules in a bioreactor containing a cell culture. For example, the training dataset may include measurements of the following examples of parameters of the second molecule manufacturing process: amount of carbon dioxide sparged into the cell culture, amount of air sparged into the cell culture, amount of oxygen sparged into the cell culture, pH primary, base total, agitation control output, back pressure, total volume of the cell culture, pH output, temperature of the cell culture, the temperature of the bioreactor/vessel shell, temperature output of a bioreactor/vessel shell controller, culture duration, osmolality, PCV, amount or concentration in the cell culture of dO₂, glucose, lactate, sodium, ammonium, carbon dioxide, O₂, and/or the like. In some instances, the training dataset can have additional, or less, manufacturing process parameters than listed here. Further, the training dataset may also include the viabilities of the cells that produce the second molecules. As such, the training dataset may include records of manufacturing process parameters and associated cell viabilities related to the manufacturing of the second molecules. In some instances, the manufacturing process parameters of the training dataset may be measured or obtained as described above with reference to the initial parameter set 106 of FIG. 1 (e.g., using sensors, probes, controllers, a combination thereof, etc.).

At step 204, the training dataset may be pre-processed to, for example, remove one or more manufacturing process parameters that have low contribution to the training of the machine learning model. For instance, all but one manufacturing process parameter of a subset of manufacturing process parameters can be redundant because the information that may be obtained from any one manufacturing process parameter of the subset for training the machine learning model may be at least substantially similar to the information that may be obtained from any other manufacturing process parameter(s) of the subset (e.g., and as such all but one may be discarded from the training dataset). As another example, a manufacturing process parameter may have low importance or contribution for training the machine learning model to predict cell viabilities. For instance, the manufacturing process parameter may have little or no correlation with cell viabilities. In such cases, the training dataset may be pre-processed using one or more of the afore-discussed feature selection technique to remove the low-importance manufacturing process parameters and reduce the training dataset. Examples of said feature selection techniques include filter methods, wrapper methods, or combination thereof.

For example, filter methods utilizing Fisher score may be applied to the training dataset to rank the manufacturing process parameters in order of their importance or contribution to the training of the machine learning model. Further, filter methods utilizing correlation coefficients may be used to calculate the correlation coefficients between the manufacturing process parameters of the training dataset to identify redundancies in the training dataset. As another example, wrapper methods may be used to form one or more subsets of the manufacturing process parameters of the training dataset and evaluate the performance of the machine learning model in predicting the cell viability when these subsets are provided as input. In various embodiments, these methods may be applied to the training dataset to remove manufacturing process parameters that may be duplicative/redundant, low-importance (e.g., contribute little or none to the training of the machine learning model) and generate a reduced training dataset. It is to be understood are these are non-limiting examples and any feature selection methods can be applied to the training dataset of manufacturing process parameters to reduce the number of manufacturing process parameters therein and generate a reduced training dataset. After the pre-processing of the training dataset, in various embodiments, the reduced training dataset may include the remaining manufacturing process parameters and the cell viability of the (initial) training dataset.

In various embodiments, it may be determined that, of the examples of parameters in the training dataset that are associated with the process for manufacturing the second molecules, the reduced training dataset (or any training dataset encompassed herein) may include the following nine examples of manufacturing process parameters: amount of carbon dioxide sparged into the cell culture, amount of air sparged into the cell culture, amount of oxygen sparged into the cell culture, pH primary (pH of the cell culture), amount or concentration of dO₂ in the bioreactor, base total (total base added into the cell culture during the molecule manufacturing process), total volume of the cell culture, temperature of the cell culture, and culture duration/time elapsed (time elapsed since the initiation of the molecule manufacturing process). In various embodiments, this reduced list of manufacturing process parameters may be different based on the second molecules. For example, the reduced training dataset can have less or more number of same or different manufacturing process parameters as the manufacturing process parameters listed immediately above for different types of the second molecules.

In some embodiments, any training dataset for any machine learning model may comprise data from a parameter consisting of, consisting essentially of, or comprising amount of carbon dioxide sparged into the cell culture. In some embodiments, any training dataset may comprise data from a parameter consisting of, consisting essentially of, or comprising amount of air sparged into the cell culture. In some embodiments, any training dataset may comprise data from a parameter consisting of, consisting essentially of, or comprising amount of oxygen sparged into the cell culture. In some embodiments, any training dataset may comprise data from a parameter consisting of, consisting essentially of, or comprising pH primary of the cell culture. In some embodiments, any training dataset may comprise data from a parameter consisting of, consisting essentially of, or comprising amount or concentration of dO₂ in the bioreactor. In some embodiments, any training dataset may comprise data from a parameter consisting of, consisting essentially of, or comprising base total of the cell culture. In some embodiments, any training dataset may comprise data from a parameter consisting of, consisting essentially of, or comprising total volume of the cell culture. In some embodiments, any training dataset may comprise data from a parameter consisting of, consisting essentially of, or comprising temperature of the cell culture. In some embodiments, any training dataset may comprise data from a parameter consisting of, consisting essentially of, or comprising time elapsed since the initiation of the molecule manufacturing process.

In various embodiments, one or multiple manufacturing process parameters may be analyzed for a trained machine learning model to generate an indicator of cell viability for any cell culture. In specific embodiments, a manufacturing process parameter comprises at least time elapsed since an initiation of the biomolecule manufacturing process, or a manufacturing process parameter does not comprise time elapsed since an initiation of the biomolecule manufacturing process. In specific embodiments, a manufacturing process parameter comprises at least total base added into the cell culture during the biomolecule manufacturing process, or a manufacturing process parameter does not comprise total base added into the cell culture during the biomolecule manufacturing process. In specific embodiments, a manufacturing process parameter comprises at least total volume of the cell culture in the bioreactor, or a manufacturing process parameter does not comprise total volume of the cell culture in the bioreactor. In specific embodiments, a manufacturing process parameter comprises at least amount of air sparged into the cell culture, or a manufacturing process parameter does not comprise amount of air sparged into the cell culture. In specific embodiments, a manufacturing process parameter comprises at least an amount of dissolved oxygen in the cell culture, or a manufacturing process parameter does not comprise an amount of dissolved oxygen in the cell culture. In specific embodiments, a manufacturing process parameter comprises at least pH of the cell culture, or a manufacturing process parameter does not comprise pH of the cell culture. In specific embodiments, a manufacturing process parameter comprises at least temperature of the cell culture, or a manufacturing process parameter does not comprise temperature of the cell culture.

In various embodiments, a training data set for a machine learning model comprises, consists of, or consists essentially of 1, 2, 3, 4, 5, 6, or 7 of any of the following: time elapsed since an initiation of the biomolecule manufacturing process; total base added into the cell culture during the biomolecule manufacturing process; total volume of the cell culture in the bioreactor; amount of air sparged into the cell culture; amount of dissolved oxygen in the cell culture; pH of the cell culture; and a temperature of the cell culture.

At step 206, in various embodiments, the reduced training dataset may be used to train the machine learning model to predict the viability of the cells that produce the second molecules. That is, the examples of nine manufacturing process parameters (or any subset thereof) and the cell viability associated therewith may be provided to the machine learning model to train the machine learning model to predict the cell viability based on an analysis of the provided nine manufacturing process parameters. For example, when a neural network is used to implement the machine learning model, the nine manufacturing process parameters may be provided to the neural network as input values. In such cases, the training of the neural network may include the iterative adjustments of weights assigned to the input values until the cell viability predicted by the neural network matches or falls within an acceptable range of the cell viability of the reduced training dataset associated with the nine manufacturing process parameters. In some instances, one may also use the initial training dataset (e.g., with the twenty one manufacturing process parameters, or a subset thereof) to train the machine learning model.

In various embodiments, once the machine learning model is trained, the effect of the manufacturing process parameters of the reduced training dataset on the prediction of the machine learning model may be computed. In some instances, the effect of a manufacturing process parameter on the prediction of the machine learning model may be quantified based on the correlation of the manufacturing process parameter with the predicted cell viability. For example, for the foregoing reduced training dataset including the nine examples of manufacturing process parameters, the effect of each of the manufacturing process parameters on the predicted cell viability may be determined by computing the correlation of the manufacturing process parameters with the predicted cell viability. In some instances, such computation may indicate the order of effect the manufacturing process parameters have on the predicted cell viability as discussed below.

In some instances, the correlation can be expressed by a Pearson correlation coefficient relating the correlation between the manufacturing process parameters and the predicted cell viability. That is, the order of effect of the manufacturing process parameters on the cell viability that is predicted by the machine learning model can be established by ordering or ranking the Pearson correlation coefficients of each manufacturing process parameters with the cell viability. In some instances, the order of effect of the manufacturing process parameters on the cell viability may be generated using machine learning techniques or methods that provide explanations about the relationship between the features that are input into machine learning models and the behavior or prediction of the machine learning models. For example, the technique permutation feature importance can be used to evaluate the importance of the manufacturing process parameters on the cell viability, and as such be used to generate the order of effect of the manufacturing process parameters on the cell viability. Other techniques that can be used to generate the order of effect include partial dependence plots (PDP) and individual conditional expectation (ICE) plots that show the dependence between the manufacturing process parameters and the cell viability. Further, the order of effect can be generated using the technique Shapley Additive exPlanations (SHAP) that computes the contribution of each manufacturing process parameter to the prediction of the cell viability.

Once the machine learning model is trained to predict cell viability using a training dataset associated with the second molecules, in various embodiments, the trained machine learning model may be used to predict viability of cells that produce the first molecules (e.g., which may be the same or different from any of the second molecules) in a cell culture in a bioreactor. To do so, at step 208, one or more manufacturing process parameters related to the process of manufacturing the first molecules in the cell culture in the bioreactor may be measured or otherwise obtained (e.g., using sensors, probes, controllers, a combination thereof, etc., and/or retrieved from databases) and provided to the trained machine learning model as input. In some instances, at step 210, the trained machine learning model may analyze the input data to predict viability of the cells that produce the first molecules in the cell culture in the bioreactor. For example, the trained machine learning model may generate an indicator (e.g., percentage, ratio, etc.) of cell viability of the cells based on the analysis.

For example, as discussed above, a training dataset that includes records of manufacturing process parameters and associated cell viability related to one or more first molecules (e.g., monoclonal antibodies) may be used to train a machine learning model. Then, in specific embodiments the manufacturing process parameters of the cell culture and bioreactor in which second molecules (for e.g. monoclonal or complex antibody or antibody fragment) are being manufactured may be measured and provided to the trained machine learning model as input for analysis to predict the viability of the cells that produce the second molecules. The second molecules can be the same as or different from any of the one or more first molecules. The input manufacturing process parameters may be measured or otherwise obtained using sensors, probes, controllers, a combination thereof, etc., that may be operationally coupled to the bioreactor. In some instances, the input manufacturing process parameters may alternatively or additionally be retrieved from databases. In such cases, the trained machine learning model may then analyze the input manufacturing process parameters and predict or generate an indicator of viability of cells that produce the second molecules in the cell culture in the bioreactor.

As example illustrations, the inventors of the instant disclosure have employed the workflow of process 200 of FIG. 2 to train machine learning models to predict indicators of cell viability of cell cultures that produce six examples of biomolecules or monoclonal antibodies (referred hereafter as “molecules 1”, “molecules 2”, “molecules 3”, “molecules 4”, “molecules 5”, and “molecules 6”). In particular, machine learning models were trained using records of manufacturing process parameters and associated cell viability using stable, engineered CHO cells that were engineered to produced the six biomolecules or monoclonal antibodies, and then used to predict indicators of cell viability of cell culture that produce the respective molecules. Fed-batch culture was used to culture the CHO cells to produce the biomolecules or monoclonal antibodies. These methods are described in Li et al., Cell culture processes for monoclonal antibody production, mAbs 2:5, 466-477; September/October 2010; Landes Bioscience; the contents of which are hereby incorporated by reference. Initially, a reduced training dataset related to biomolecules 1 was generated by pairing the nine manufacturing process parameters listed above (e.g., measured during a process for manufacturing biomolecules 1) and cell viability of the cells that produced biomolecules 1 during the manufacturing process. In some instances, the cell viability in the training dataset may be measured by manually sampling the cell culture. The reduced training dataset was then used to train a machine learning model to predict the measured cell viabilities. The trained machine learning model was then used to predict cell viability of cell cultures of manufacturing processes designed to produce biomolecules 1 (at least substantially similar steps were repeated to train machine learning models to predict cell viabilities of cell cultures of manufacturing processes designed to produce biomolecules 2, biomolecules 3, biomolecules 4, biomolecules 5, and biomolecules 6). In some instances, the reduced training datasets corresponding to multiple biomolecules (e.g., all six biomolecules or monoclonal antibodies, sometimes referred as “generic molecule”) were combined into a single training dataset, which is then used to train a machine learning model. The trained machine learning model was then used to predict or generate indicators of cell viabilities of cell cultures of manufacturing processes designed to produce any of the multiple biomolecules. In some embodiments, five of the six biomolecules, four of the six biomolecules, three of the six biomolecules, or two of the six biomolecules could have been utilized to have combined into a single training dataset.

In various embodiments, the orders of effect of the nine manufacturing process parameters on the indicators of cell viabilities of the cells producing the six biomolecules (monoclonal antibodies) can be generated as discussed above. In at least some instances, it was found that a first set of the manufacturing process parameters may have higher effect on the predicted indicator of cell viability than a second set of the manufacturing process parameters for the six biomolecules, where the first set comprises, as examples, time elapsed since an initiation of the biomolecule manufacturing process, total base added into the cell culture during the biomolecule manufacturing process, and a total volume of the cell culture in the bioreactor culture, and the second set comprises an amount of air sparged into the cell culture, an amount of dissolved oxygen in the cell culture, a pH of the cell culture, and a temperature of the cell culture. That is, in some embodiments, any one of the manufacturing process parameters of the first set may have higher effect on (e.g., higher correlation with) the predicted indicator of cell viability than any of the manufacturing process parameters of the second set. It is to be noted that the contents of the first set and/or the second set may depend on the biomolecules being produced by the cells. That is, for example, the contents of the first set and/or the second set for any one of biomolecules 1, biomolecules 2, biomolecules 3, biomolecules 4, biomolecules 5, or biomolecules 6 may be different from the contents of the first set and/or the second set for any of the other biomolecules or combinations thereof.

In some embodiments, the first set of manufacturing process parameters that have higher effect on the indicator of cell viability of the cells that produce biomolecules 5 than the second set of manufacturing process parameters further includes the amount of oxygen sparged into the cell culture. This first set of manufacturing process parameters for biomolecules 5 is shown below in Table 1 (reproduced from above), listed in order of effect on the indicator of cell viability. Table 1=

| Serial No. | Manufacturing process parameter |
|---|---|
| 1 | the time elapsed since the |
| | initiation of the biomolecule |
| | manufacturing process |
| 2 | the amount of oxygen sparged into |
| | the cell culture |
| 3 | the total volume of the cell culture |
| 4 | the total base added into the cell |
| | culture during the biomolecule |
| | manufacturing process |

The second set of manufacturing process parameters for biomolecules 5 further includes the amount of carbon dioxide sparged into the cell culture and is shown below in Table 2 (reproduced from above), listed in order of effect on the indicator of cell viability. Table 2=

| Serial No. | Manufacturing process parameter |
|---|---|
| 1 | the amount of carbon dioxide |
| | sparged into the cell culture |
| 2 | the amount of dissolved oxygen in |
| | the cell culture |
| 3 | the pH of the cell culture |
| 4 | the amount of air sparged into the |
| | cell culture |
| 5 | the temperature of the cell culture |

The first set of manufacturing process parameters for biomolecules 1, biomolecules 2, or biomolecules 3 is shown below in Table 3 (reproduced from above), listed in order of effect on the indicator of cell viability. Table 3=

| Serial No. | Manufacturing process parameter |
|---|---|
| 1 | the time elapsed since the |
| | initiation of the biomolecule |
| | manufacturing process |
| 2 | the total base added into the cell |
| | culture during the biomolecule |
| | manufacturing process |
| 3 | the total volume of the cell culture |

The second set of manufacturing process parameters for biomolecules 3 is shown below in Table 4 (reproduced from above), listed in order of effect on the indicator of cell viability. Table 4=

| Serial No. | Manufacturing process parameter |
|---|---|
| 1 | the amount of oxygen sparged into |
| | the cell culture |
| 2 | the amount of carbon dioxide |
| | sparged into the cell culture |
| 3 | the temperature of the cell culture |
| 4 | the amount of air sparged into the |
| | cell culture |
| 5 | the pH of the cell culture |
| 6 | the amount of dissolved oxygen in |
| | the cell culture |

The second set of manufacturing process parameters for biomolecules 2 is shown below in Table 5 (reproduced from above), listed in order of effect on the indicator of cell viability. Table 5=

| Serial No. | Manufacturing process parameter |
|---|---|
| 1 | the amount of air sparged into the |
| | cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of carbon dioxide |
| | sparged into the cell culture |

The second set of manufacturing process parameters for biomolecules 1 is shown below in Table 6 (reproduced from above), listed in order of effect on the indicator of cell viability. Table 6=

| Serial No. | Manufacturing process parameter |
|---|---|
| 1 | the pH of the cell culture |
| 2 | the amount of carbon dioxide |
| | sparged into the cell culture |
| 3 | the temperature of the cell culture |
| 4 | the amount of oxygen sparged into |
| | the cell culture |
| 5 | the amount of air sparged into the |
| | cell culture |
| 6 | the amount of dissolved oxygen in |
| | the cell culture |

The first set of manufacturing process parameters for biomolecules 4 or biomolecules 6 further includes the amount of carbon dioxide sparged into the cell culture. This first set of manufacturing process parameters for biomolecules 4 or biomolecules 6 is shown below in Table 7 (reproduced from above), listed in order of effect on the indicator of cell viability. Table 7=

| Serial No. | Manufacturing process parameter |
|---|---|
| 1 | the time elapsed since the |
| | initiation of the biomolecule |
| | manufacturing process |
| 2 | the amount of carbon dioxide |
| | sparged into the cell culture |
| 3 | the total base added into the cell |
| | culture during the biomolecule |
| | manufacturing process |
| 4 | the total volume of the cell culture |

The second set of manufacturing process parameters for biomolecules 4 further includes the amount of oxygen sparged into the cell culture, and is shown below in Table 8 (reproduced from above), listed in order of effect on the indicator of cell viability. Table 8=

| Serial No. | Manufacturing process parameter |
|---|---|
| 1 | the amount of air sparged into the |
| | cell culture |
| 2 | the amount of dissolved oxygen in |
| | the cell culture |
| 3 | the temperature of the cell culture |
| 4 | the pH of the cell culture |
| 5 | the amount of oxygen sparged into |
| | the cell culture |

The second set of manufacturing process parameters for biomolecules 6 further includes the amount of oxygen sparged into the cell culture, and is shown below in Table 9 (reproduced from above), listed in order of effect on the indicator of cell viability. Table 9=

| Serial No. | Manufacturing process parameter |
|---|---|
| 1 | the temperature of the cell culture |
| 2 | the amount of oxygen sparged into |
| 3 | the pH of the cell culture |
| 4 | the amount of air sparged into the |
| | cell culture |
| 5 | the cell culture the amount of |
| | dissolved oxygen in the cell |
| | culture |

In various embodiments, as discussed above, the training datasets corresponding to the six biomolecules may be combined into a single training dataset (“generic dataset”) and may be used to train a machine learning model. The trained machine learning model may be used to predict or generate an indicator of cell viability of cell culture that produce any one of the six biomolecules, and the order of effect of the nine manufacturing process parameters on the indicator of cell viability may be generated as discussed above. In such cases, the nine manufacturing process parameters for this generic case listed in order of effect on the indicator of cell viability are shown in Table 12 below. A first set of three manufacturing process parameters having higher order of effect on the indicator of cell viability than the rest six manufacturing process parameters are shown in Table 10 below (reproduced from above), listed in order of effect on the indicator of cell viability. Table 10=

| Serial No. | Manufacturing process parameter |
|---|---|
| 1 | the time elapsed since the |
| | initiation of the biomolecule |
| | manufacturing process |
| 2 | the total volume of the cell culture |
| 3 | the total base added into the cell |
| | culture during the biomolecule |
| | manufacturing process |

A second set of the remaining six manufacturing process parameters are shown in table 11 below, listed in order of effect on the indicator of cell viability. Table 11=

| Serial No. | Manufacturing process parameter |
|---|---|
| 1 | the amount of carbon dioxide |
| | sparged into the cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of air sparged into the |
| | cell culture |

In Tables 1-11, “Serial No.” is an index denoting the order of effect of the corresponding manufacturing process parameter indexed by that serial number on the indicator of cell viability that is predicted by the machine learning model. For example, for biomolecules 5, Tables 1 and 2 in combination indicate that the time elapsed since the manufacturing of the biomolecules commenced (“culture duration” or “time elapsed”) has the highest effect on the indicator of cell viability than any of the other manufacturing process parameters considered.

Once a machine learning model is trained to predict or generate an indicator of cell viability (e.g., using a training dataset that includes the nine manufacturing process parameters), a selected number of the nine manufacturing process parameters related to a biomolecule manufacturing process may be measured and provided to the trained machine learning model to predict the indicator of cell viability of cells that produce the biomolecules during the manufacturing process. In some instances, the selected number of the nine manufacturing process parameters can be at least three, at least four, at least five, at least six, at least seven, at least eight, or all nine of the manufacturing process parameters. When a first set of the nine manufacturing process parameters has higher order of effect on the indicator of cell viability than that of a second set of the nine manufacturing process parameters, the selected number of the nine manufacturing process parameters can be drawn from one or both of the first set or second set. In some instances, the number of input manufacturing process parameters can depend on the desired level of accuracy of the trained machine learning model's prediction of the indicator of cell viability.

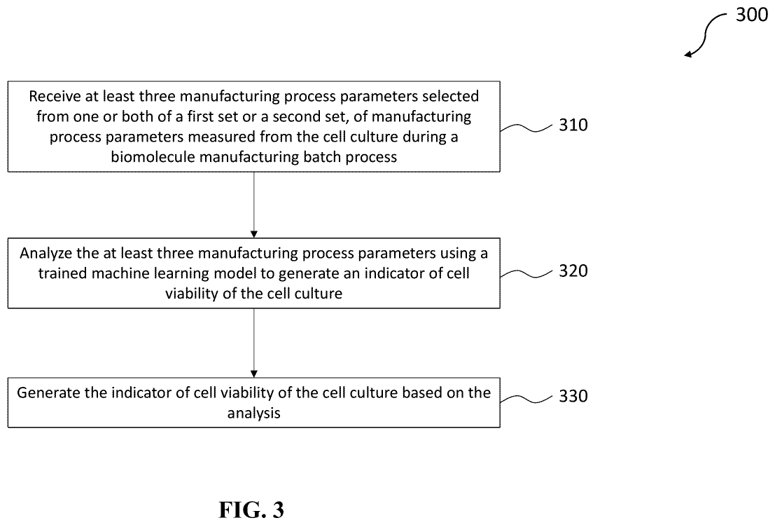

FIG. 3 shows a flowchart of a machine learning model-based process 300 for predicting cell viability of a cell culture in a bioreactor during a biomolecule manufacturing process, in accordance with various embodiments. In various embodiments, method 300 is implemented using a system, such as, the cell viability prediction system 100 of FIG. 1 to predict indicator of cell viability 112 of cells that produce biomolecules during a biomolecule manufacturing batch process.

At step 310, at least three manufacturing process parameters selected from one or both of a first set of manufacturing process parameters or a second set of manufacturing process parameters are received. In some instances, the manufacturing process parameters are measured from a cell culture during the biomolecule manufacturing process. In various embodiments, any of the first set of manufacturing process parameters has an order of effect on the indicator of cell viability of the cell culture that is higher than that of any of the second set of manufacturing process parameters. In some instances, the biomolecules include monoclonal antibodies or antibody fragments.

In various embodiments, the first set of process parameters includes time elapsed since an initiation of the biomolecule manufacturing process, total base added into the cell culture during the biomolecule manufacturing process, and a total volume of the cell culture in the bioreactor. In various embodiments, the second set of process parameters includes an amount of air sparged into the cell culture, an amount of dissolved oxygen in the cell culture, a pH of the cell culture, and a temperature of the cell culture.

At step 320, the at least three manufacturing process parameters are analyzed using a trained machine learning model to generate an indicator of cell viability of the cell culture.

At step 330, the indicator of cell viability of the cell culture may be generated by the trained machine learning model based on the analysis.

In various embodiments of method 300, the machine learning model can be a neural network, or a decision-tree based machine learning model. As described previously, the machine learning model 110 can also be and is not limited to a neural network, a decision tree, a random forest, a support vector machine, a Bayesian network, a regression tree, and/or the like and these are considered as embodiments of method 300. Further, the neural network can be a deep neural network, a convolutional neural network (CNN), an artificial neural network (ANN), a recurrent neural network (RNN), a modular neural network (MNN), a residual neural network (RNN), an ordinary differential equations neural networks (neural-ODE), a squeeze and excitation embedded neural network, a MobileNet, etc. The ANN can be a long short-term memory (LSTM) neural network. The regression tree can be a gradient boosting machine (GBM) model (e.g., XGBoost). In some instances, the machine learning model 110 can be a probabilistic graphical model with a Bayesian network. Further, the machine learning model can be trained with manufacturing process training records that include the first and second sets of manufacturing process parameters and the indicator of cell viability of the cell culture.

In various embodiments of method 300, the at least one manufacturing process parameter of the one or both of the first set of manufacturing process parameters or the second set of manufacturing process parameters is measured by a sensor operationally connected to the bioreactor. In such instances, the at least one manufacturing process parameter is the pH of the cell culture, the temperature of the cell culture, the amount of dissolved oxygen in the cell culture, or the total volume of the cell culture, and the sensor is a temperature probe, a dissolved oxygen probe, a pH probe, or a scale configured to weigh the cell culture, respectively, disposed within the bioreactor.

In various embodiments of method 300, the at least one manufacturing process parameter of the one or both of the first set of manufacturing process parameters or the second set of manufacturing process parameters is an output of a controller operationally connected to the bioreactor. In such instances, the at least one manufacturing process parameter is the amount of air sparged into the cell culture, the amount of carbon dioxide sparged into the cell culture, or the amount of oxygen sparged into the cell culture, and the controller is an air flow controller configured to control flow of the air sparged into the cell culture, the carbon dioxide sparged into the cell culture, or the oxygen sparged into the cell culture, respectively.

### IV. Pre-Processing of Training Dataset

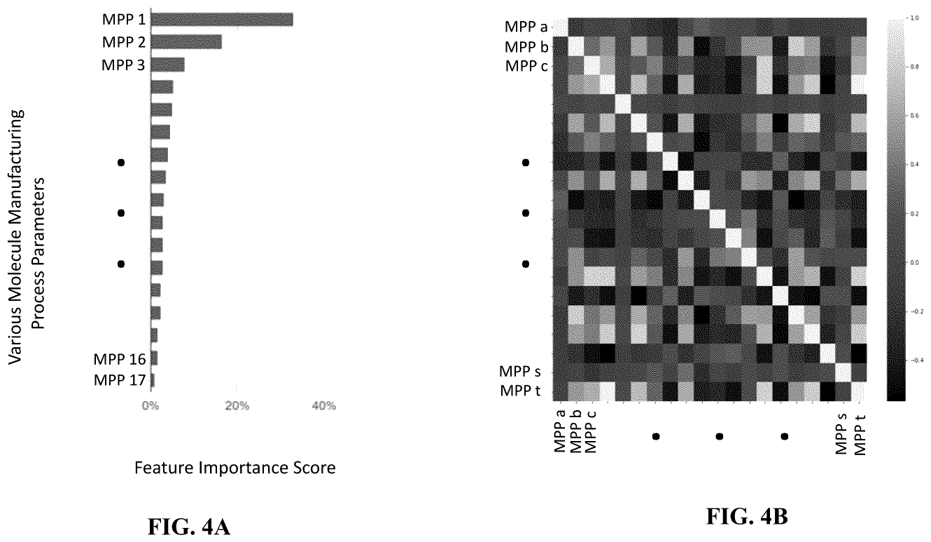

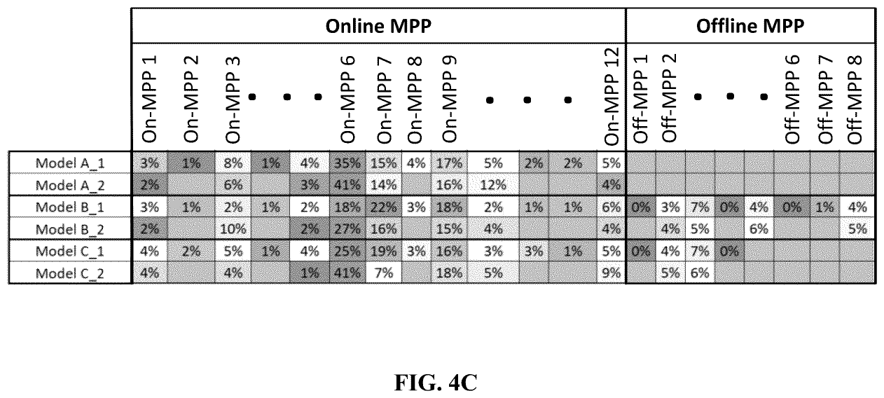

FIGS. 4A-4C show a bar graph (FIG. 4A), a heatmap (FIG. 4B), and a table (FIG. 4C) illustrating the identification of biomolecule manufacturing process parameters for use in training a machine learning model to predict viability of cells that produce the biomolecules, in accordance with various embodiments. In various embodiments, as discussed above, an initial set of manufacturing process parameters related to a process for manufacturing biomolecules in a bioreactor may be measured (e.g., using probes, sensors, controllers, etc.) or otherwise obtained (e.g., retrieved from a database) to include into a training dataset for training the machine learning model. The manufacturing process parameters include amount of carbon dioxide sparged into the cell culture, amount of air sparged into the cell culture, amount of oxygen sparged into the cell culture, pH primary, amount or concentration of dissolved oxygen (dO₂) in the bioreactor, base total, agitation control output, back pressure, total volume of the cell culture, pH output, temperature of the cell culture, the temperature of the bioreactor/vessel shell (“jacket temperature”), temperature output of a bioreactor/vessel shell controller, culture duration/elapsed time, packed cell volume (PCV), duration days/elapsed time, osmolality, and concentrations of glucose, lactate, sodium, ammonium, carbon dioxide, oxygen (02), etc., in the cell culture, and/or the like, related to the process of manufacturing the biomolecules in the bioreactor.

FIG. 4A illustrates a bar graph of feature importance scores (X-axis) in relation to various MPPs or Features numbered 1 through 17. MPP stands for “molecule manufacturing process parameter” that include but are not limited to features such as pH, temperature, the amount of dissolved oxygen in the cell culture, and a total volume of the cell culture; or features that are measured from instruments/sensors such as temperature probe, dissolved oxygen probe, pH probe, a liquid level imaging sensor, scale configured to weigh the cell culture, etc. In FIG. 4A, the feature Importance Score is the relative score (weight) assigned to each feature when building a predictive model. In FIG. 4A, a feature is synonymous with a molecule manufacturing process parameter or MPP. Features or MPP are inputs that are used as predictors in the model. So, in particular aspects the higher the importance score, the larger the impact of the MPP on the model.

In some instances, with reference to FIG. 4A, one may wish to reduce this initial set of manufacturing process parameters to remove those manufacturing process parameters that have little or no contribution towards the training the machine learning model. In such cases, the importance of the manufacturing process parameters of the initial set with respect to the training of the machine learning model may be computed, and the initial set narrowed down to a reduced set of manufacturing process parameters based on the computed measure of importance. For instance, the manufacturing process parameters having associated therewith an importance score below a threshold importance score may be discarded and not included in the training dataset (i.e., the initial training dataset may be reduced).

In some instances, the importance of the manufacturing process parameters may be computed using a feature selection technique. For example, the afore-mentioned filter methods may be applied to the manufacturing process parameters of the initial set to quantify the importance of the manufacturing process parameters for training the machine learning model. For instance, Fisher scores of the manufacturing process parameters may be computed to quantify the importance of the manufacturing process parameters for training the machine learning model. As another example, Pearson correlation coefficients of the manufacturing process parameters with output of the machine learning model (e.g., indicator of cell viability) may also be used to quantify the importance of the manufacturing process parameters for training the machine learning model. Statistical techniques utilizing information gain/mutual information, variance threshold, etc., may also be used to determine the importance of the manufacturing process parameters. FIG. 4A shows an example embodiment of a bar graph of Pearson correlation coefficients comparing the importance of the afore-mentioned seventeen manufacturing process parameters (MPPs) for use in training a machine learning model to predict the viability of the cells that produce the biomolecules.

In various embodiments, filter methods employing correlation coefficients may be applied to the manufacturing process parameters to characterize the correlations between any two of the manufacturing process parameters. And because highly correlated manufacturing process parameters contribute largely similar information towards the training of the machine learning model, the correlations can be used to remove the redundancy from the training dataset of manufacturing process parameters. FIG. 4B shows a heatmap of correlations between multiple manufacturing process parameters. FIG. 4B: is a correlation matrix/heatmap which correlates any two features (MPPs). The lighter the tile color, the stronger the correlation; and conversely, the darker the tile, the weaker the correlation. The heatmap is a simplified visual depiction of correlation between features (MPP) that facilitates the process of selecting features as inputs for the ML model. In order to build the model, if two features share a high correlation (typically above a certain threshold), only one feature is added as an input into the model. As the process of model building is iterative, inputs may be eliminated if they negatively impact the “fit” or predictability of the model. In such cases, when the heatmap indicates a pair of manufacturing process parameters are highly correlated (e.g., their correlation value exceeds a correlation threshold), one of the pair may be discarded and not included in the training dataset, reducing redundancy in the training dataset and as a result associated computational and model training inefficiencies.

In various embodiments, FIG. 4C shows an example application of wrapper methods of feature selection where subsets of the initial set of manufacturing process parameters are formed and used to determine the importance of a manufacturing process parameter to the prediction of a machine learning model. The correlations between the selected manufacturing process parameters and the prediction of the machine learning model (e.g., the cell viability) are computed to determine those manufacturing process parameters that are strongly correlated with the predicted cell viability. In some instances, such strongly correlated manufacturing process parameters may be included to the reduced training dataset that may then be used to train the machine learning model to predict cell viability.

For example, with reference to FIG. 4C, the various “Models” refer to machine learning models that are provided input datasets of manufacturing process parameters excluding the grayed out parameters. For instance, the input dataset of manufacturing process parameters that is provided to the machine learning model “Model A_1” excludes all the offline manufacturing process parameters (Off-MPP1 to Off-MPP 8), while that provided to machine learning model “Model C_1” excludes offline parameters Off-MPP 6, Off-MPP 7, and Off-MPP 8. Online and offline manufacturing process parameters (“On-MPP” and “Off-MPP”) refer to those parameters that are measured or obtained from the bioreactor in real time (e.g., using probes, controllers, etc.) and those parameters that are measured or obtained otherwise (e.g., manually using cell culture sampled from the bioreactor), respectively. The relative correlations of the included parameters with the cell viability predicted by the respective machine learning models may then be computed. In some instances, those parameters that have high correlation with the prediction of the model may then be included into the training dataset for training the machine learning model. For example, FIG. 4C shows that the manufacturing process parameters On-MPP 6, On-MPP 7, and On-MPP 8 have relatively large correlation with the cell viability predictions of both “Model A_1” and “Model C_1”. As such, these manufacturing process parameters may be included in the reduced training dataset to be used for training the machine learning model.

### V. Training and Testing of Machine Learning Model

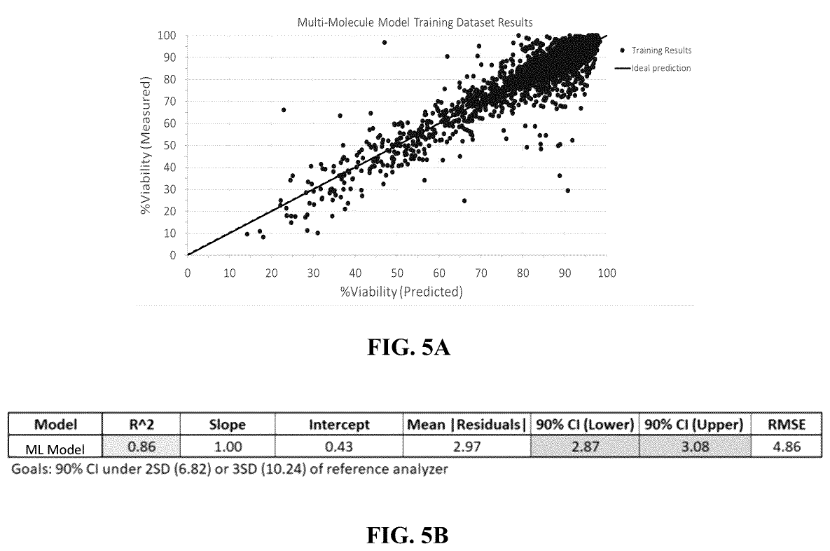

As discussed above, in various embodiments, the training dataset for training a machine learning model includes records of manufacturing process parameters that are related to processes for manufacturing biomolecules and viabilities of cells that produce the biomolecules. FIGS. 5A-5B show a graph (FIG. 5A) and a table (FIG. 5B) illustrating the training of a machine learning model using a training dataset comprising records of manufacturing process parameters that are related to processes for manufacturing the afore-mentioned six types of biomolecules, biomolecules 1, biomolecules 2, biomolecules 3, biomolecules 4, biomolecules 5, and biomolecules 6, and measured indicators of cell viabilities of cells that produce the biomolecules, in accordance with various embodiments. The manufacturing process parameters were obtained using sensors, probes, controllers, etc., as discussed above, and the cell viabilities were measured by sampling the cell cultures in the bioreactors in which the biomolecules were manufactured. The training dataset was then compiled by combining the manufacturing process parameters and the cell viabilities that correspond to the six types of biomolecules. In some instances, training datasets can be generated using the manufacturing process parameters and cell viability records of less than all six types of biomolecules (e.g., using the records of only one, only two, only three, only four, or only five of the six types of biomolecules).

FIGS. 5A and 5B show a scatter plot and a table including details of the best fit thereof, respectively, comparing the measured indicators of cell viability for the six different types of biomolecules to the indicators of cell viability predicted by the trained machine learning model, indicating good agreement between the two for a broad range of cell viability values (e.g., and as such, the successful training of the machine learning model). FIG. 5A provides a graph of measured vs. predicted viability of cells. FIG. 5B: provides a table with a listing of performance evaluation metrics, for e.g., R2, mean, residuals, RMSE, etc.

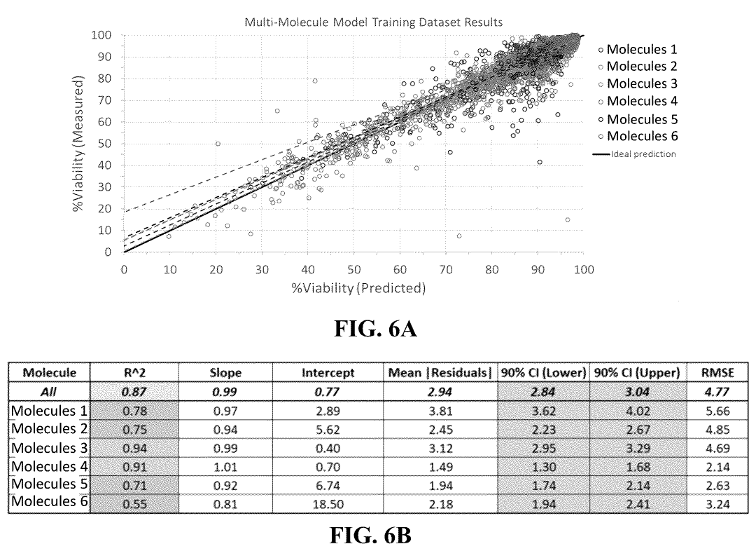

FIGS. 6A and 6B show scatter plots and a table including details of the best fits thereof, respectively, comparing measured indicators of cell viability to predicted indicators of cell viability broken down by the biomolecules the records of which were used to generate the training dataset that is used to train the machine learning model. The figures demonstrate that a training dataset that includes records of manufacturing process parameters related to processes for manufacturing the six different biomolecules (antibodies) can be used to predict viability of cells that produce any one of the six different biomolecules. It is to be understood that although the discussion herein relates to six different biomolecules, it is applicable to any number of types of biomolecules.

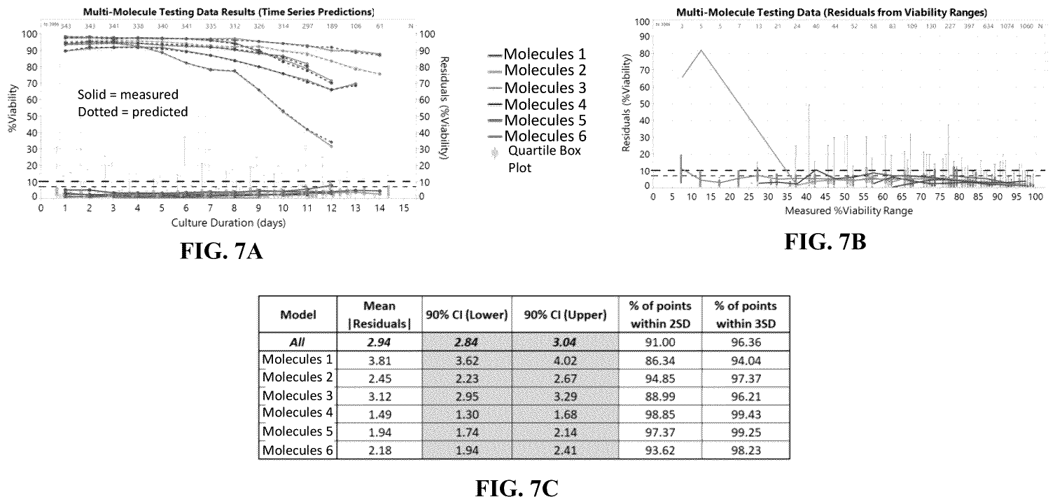

FIGS. 7A-7C show graphs (FIGS. 7A and 7B) and a table (FIG. 6C) illustrating the testing of the trained machine learning model, in accordance with various embodiments. In various embodiments, the machine learning model trained as discussed above with reference to FIGS. 6A-6B may analyze a testing dataset of manufacturing process parameters that are related to the processes for manufacturing the six biomolecules. FIGS. 7A-7C illustrate that the trained machine learning model can predict viabilities of the cells that produce the biomolecules with significant accuracy. For example, except for one errant data point, the residuals, i.e., the differences between the measured and the predicted cell viabilities, are low for a wide range of the cell viabilities, as shown in FIG. 7B. Further, FIG. 7A shows that the trained machine learning model can predict the cell viabilities with significant accuracy for an extended time period (e.g., culture duration of the biomolecules manufacturing processes measured in days).

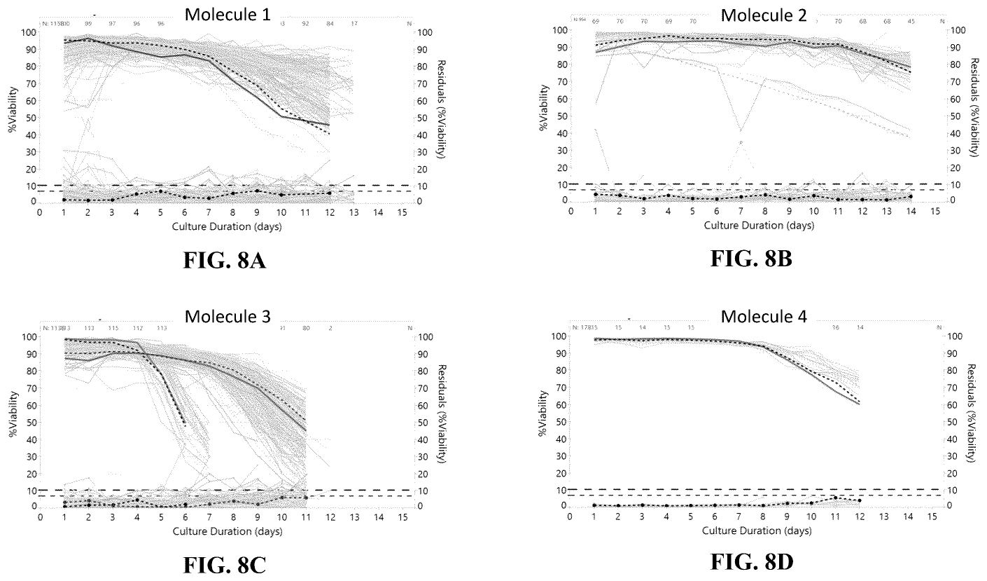

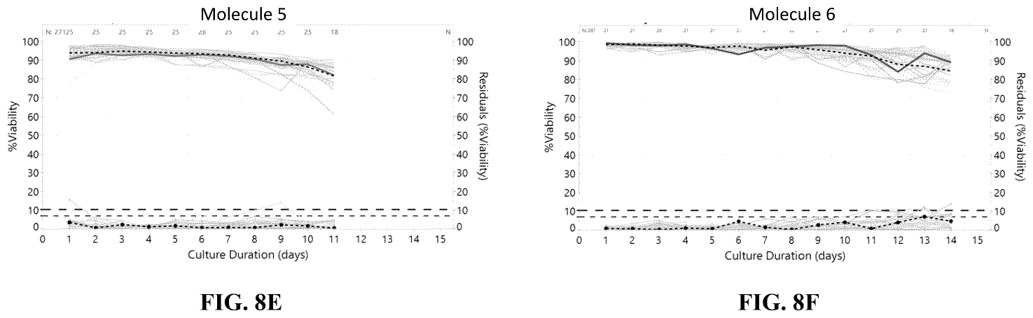

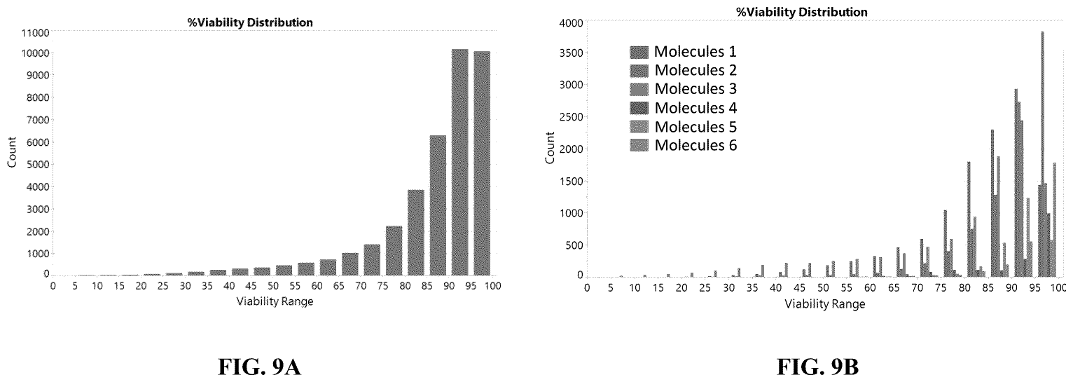

FIGS. 8A-8F show graphs illustrating cell viability predictions of a machine learning model for multiple types of biomolecules produced by a process for manufacturing the biomolecules, in accordance with various embodiments. In various embodiments, a portion of the training dataset (e.g., ten to 20 batches of the manufacturing process parameters and associated cell viabilities) were excluded from the training dataset for later use to test the machine learning model after the machine learning model is trained with the remaining training dataset. The graphs show the predicted indicators of cell viabilities and residuals for the six biomolecules (monoclonal antibodies) referenced with respect to FIGS. 6A-6B and 7A-7C over the culture duration time period (the bold and dotted lines show measure and predicted results, respectively, for a single batch). It is to be noted that the trained machine learning model predicts the cell viability with high accuracy for different biomolecules as well as different cell viability time profiles (e.g., biomolecules 6 correspond to an indicator of cell viability that remain largely consistent throughout the culture duration (FIG. 8F) while the indicator of cell viability of biomolecules 3 drops much more quickly (FIG. 8C)). FIG. 9A shows the cell viability distribution for the batches of the training dataset of manufacturing process parameters for all six of the biomolecules, while FIG. 9B shows the cell viability distribution broken down by biomolecules.

### VI. Computer Implemented System

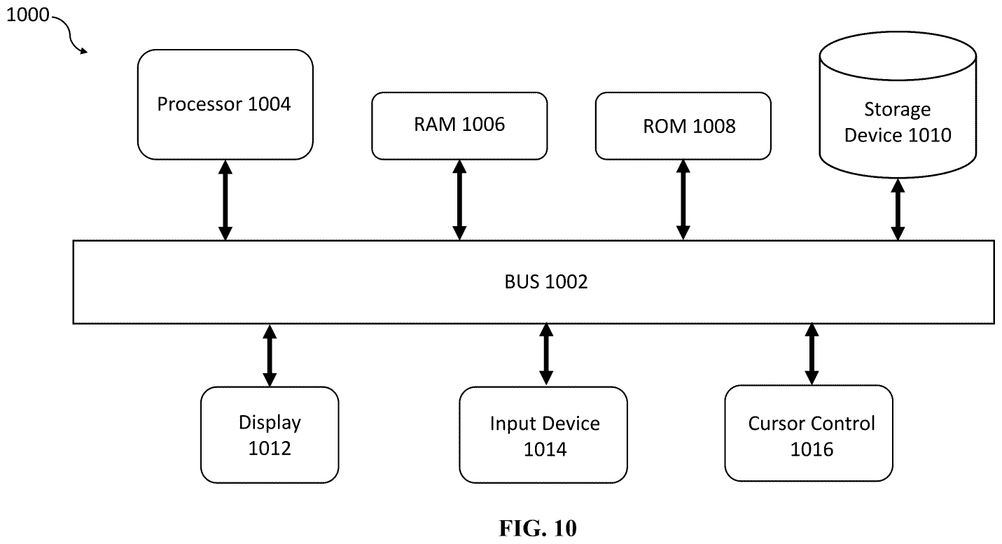

FIG. 10 is a block diagram of a computer system in accordance with various embodiments. Computer system 1000 may be an example of one implementation for cell viability prediction system 100 described above in FIG. 1. In one or more examples, computer system 1000 can include a bus 1002 or other communication mechanism for communicating information, and a processor 1004 coupled with bus 1002 for processing information. In various embodiments, computer system 1000 can also include a memory, which can be a random-access memory (RAM) 1006 or other dynamic storage device, coupled to bus 1002 for determining instructions to be executed by processor 1004. Memory also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a joystick, a trackball, a gesture input device, a gaze-based input device, or cursor direction keys for communicating direction information and command selections to processor 1004 and for controlling cursor movement on display 1012. This input device 1014 typically has two degrees of freedom in two axes, a first axis (e.g., x) and a second axis (e.g., y), that allows the device to specify positions in a plane. However, it should be understood that input devices 1014 allowing for three-dimensional (e.g., x, y and z) cursor movement are also contemplated herein.

Consistent with certain implementations of the present teachings, results can be provided by computer system 1000 in response to processor 1004 executing one or more sequences of one or more instructions contained in RAM 1006. Such instructions can be read into RAM 1006 from another computer-readable medium or computer-readable storage medium, such as storage device 1010. Execution of the sequences of instructions contained in RAM 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as RAM 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 1000 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000, whereby processor 1004 would execute the analyses and determinations provided by these engines, subject to instructions provided by any one of, or a combination of, the memory components RAM 1006, ROM, 1008, or storage device 1010 and user input provided via input device 1014.

### VII. Artificial Neural Networks

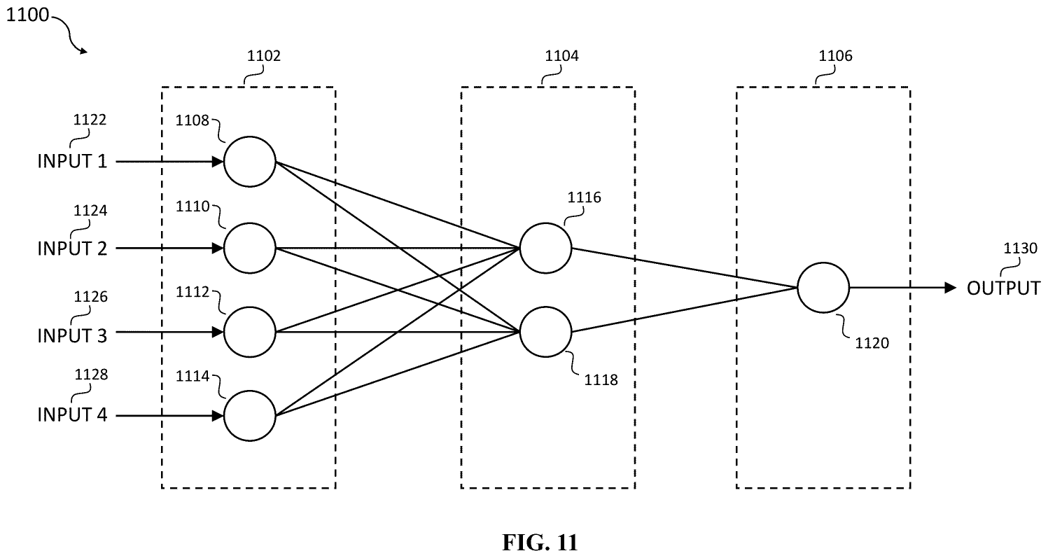

FIG. 11 illustrates an example neural network that can be used to implement a machine learning model according to various embodiments of the present disclosure. For example, in various embodiments, the machine learning model 110 can be implemented using the neural network 1100. As shown, the artificial neural network 1100 includes three layers—an input layer 1102, a hidden layer 1104, and an output layer 1106. Each of the layers 1102, 1104, and 1106 may include one or more nodes. For example, the input layer 1102 includes nodes 1108-1114, the hidden layer 1104 includes nodes 1116-1118, and the output layer 1106 includes a node 1122. In this example, each node in a layer is connected to every node in an adjacent layer. For example, the node 1108 in the input layer 1102 is connected to both of the nodes 1116, 1118 in the hidden layer 1104. Similarly, the node 1116 in the hidden layer is connected to all of the nodes 1108-1114 in the input layer 1102 and the node 1122 in the output layer 1106. Although only one hidden layer is shown for the artificial neural network 1100, it has been contemplated that the artificial neural network 1100 used to implement the machine learning model 110 may include as many hidden layers as necessary or desired.

In this example, the artificial neural network 1100 receives a set of input values 1122-1128 and produces an output value 1130. Each node in the input layer 1102 may correspond to a distinct input value. For example, nodes 1108-1114 in the input layer 1102 may correspond to input values 1122-1128, respectively. In some instances, the input values 1122-1128 may correspond to parameters or values that are provided to the machine learning model 110 as input. For example, when the artificial neural network 1100 is used to implement the machine learning model 110, each node in the input layer 1102 may correspond to the manufacturing process parameters of the reduced parameter set 108.

In some embodiments, each of the nodes 1116-1118 in the hidden layer 1104 generates a representation, which may include a mathematical computation (or algorithm) that produces a value based on the input values received from the nodes 1108-1114. The mathematical computation may include assigning different weights to each of the data values received from the nodes 1108-1114. The nodes 1116 and 1118 may include different algorithms and/or different weights assigned to the data variables from the nodes 1108-1114 such that each of the nodes 1116-1118 may produce a different value based on the same input values received from the nodes 1108-1114. In some embodiments, the weights that are initially assigned to the features (or input values) for each of the nodes 1116-1118 may be randomly generated (e.g., using a computer randomizer). The values generated by the nodes 1116 and 1118 may be used by the node 1122 in the output layer 1106 to produce an output value for the artificial neural network 1100. When the artificial neural network 1100 is used to implement the machine learning model 110, the output value 1130 produced by the artificial neural network 1100 may correspond to the cell viability 112.

The artificial neural network 1100 may be trained by using training data. For example, the training data herein may be the reduced parameter set 108 or the initial parameter set 106. By providing training data to the artificial neural network 1100, the nodes 1116-1118 in the hidden layer 1104 may be trained (adjusted) such that an optimal output is produced in the output layer 1106 based on the training data. By continuously providing different sets of training data, and penalizing the artificial neural network 1100 when the output of the artificial neural network 1100 is incorrect (e.g., when the difference between the predicted cell viability 112 and the measured cell viability exceeds some threshold), the artificial neural network 1100 (and specifically, the representations of the nodes in the hidden layer 1104) may be trained (adjusted) to improve its performance. In some instances, adjusting the artificial neural network 1100 may include adjusting the weights associated with each node in the hidden layer 1104.

Although the above discussions pertain to an artificial neural network as an example of machine learning, it is understood that other types of machine learning methods may also be suitable to implement the various aspects of the present disclosure. For example, support vector machines (SVMs) may be used to implement machine learning. SVMs are a set of related supervised learning methods used for classification and regression. A SVM training algorithm which may be a non-probabilistic binary linear classifier may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable.

Another example is a machine learning engine that employs a decision tree learning model to conduct the machine learning process. In some instances, decision tree learning models may include classification tree models, as well as regression tree models. In some embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

### VIII. Cells and Biomolecules Produced Therefrom

In particular embodiments, the cell viability of a culture, including percent cell viability of a culture, is determined utilizing systems, methods, and compositions encompassed herein. The cell viability of one or more cultures may be determined, identified, measured, assayed, calculated, assessed, predicted, quantified, projected, forecast, etc., based on systems, methods, and compositions encompassed herein. In certain embodiments, the cell viability of one or more cell cultures is an object of methods of the disclosure as an indicator of the quality and/or amount of biomolecules produced by the cells in the cell culture, including at least the majority of cells in the cell culture. In some embodiments, the cell viability of one or more cell cultures is utilized as an indirect gauge of the quantity of biomolecules produced by the cells of the cell culture, and therefore in at least some cases a gauge of the amount of biomolecules secreted by the cells and in the cell media of the cell culture. In particular cases following production of the cell culture, the biomolecules are procured from inside the cells in addition to, or alternatively to, being procured from the media of the culture itself, although both may occur.

In particular embodiments, the cells utilized in the cell culture may be of any source and any type. In specific embodiments, the cells are eukaryotic or prokaryotic. The cells may be bacterial cells, fungal cells, animal cells, insect cells, avian cells, plant cells, yeast cells, algal cells, or a mixture thereof, in some cases. The cells may or may not be mammalian cells. The cells may or may not be lower eukaryotic cells, e.g., yeast cells or filamentous fungi cells. In cases wherein the cells are bacterial cells, they may be Gram-positive cells or Gram-negative cells. In cases wherein the cells are mammalian cells, the cells may be from a human, rat, mouse, or mixture thereof. In some embodiments, the cells are allogeneic, autologous, or xenogeneic with respect to an individual.

Although in some embodiments the cells themselves are a pharmaceutical agent (whether or not they produced one or more therapeutic agents), in certain embodiments the cells produce a biomolecule that may be utilized as a pharmaceutical agent, regardless of whether or not it is secreted from the cells. Examples of biomolecules that may be produced by the cells include at least proteins (such as antibodies of any kind, including at least monoclonal antibodies or antibody fusions), recombinant proteins, peptides, amino acids, fatty acids, carbohydrates, and so forth.

In some embodiments, the cells themselves are not therapeutic but produce one or more types of biomolecules that are therapeutic. When the cells themselves are therapeutic, they may or may not produce one or more types of biomolecules, and in any case the cells themselves may be modified by the hand of man. In particular embodiments the cells are modified to express one or more heterologous molecules that by themselves may or may not be therapeutic, but in some cases the collective of the cells plus heterologous biomolecules is therapeutic. In specific embodiments, the cells may comprise an engineered (e.g., produced by the hand of man) biomolecule, including at least an engineered receptor of any kind, including an engineered chimeric antigen receptor, an engineered T cell receptor, an engineered chimeric receptor of any kind, an engineered cytokine receptor, and so forth. The engineered biomolecule may or may not be secreted from the cell. Any cell encompassed herein may comprise heterologous molecules that provide gene therapy of any kind (RNA or DNA), engineered viruses, engineered receptors, etc., and in some embodiments the cells comprise cell therapy safety measures. In some embodiments, the cells target an antigen, such as with an engineered antigen receptor, an antibody, a bispecific antibody, an engager, etc., and the antigen may be a bacterial antigen, a fungal antigen, a viral antigen, a cancer antigen (hematopoietic or of solid tumors), etc.

In particular embodiments, the cell cultures are utilized for culturing suspension cells or anchorage-dependent (adherent) cells. In some embodiments, the cells are suitable for producing pharmaceutical products and may be cellular and/or viral therapies, in at least specific embodiments. Embodiments of pharmaceutical products include at least peptides, polypeptides, nucleic acids (DNA and/or RNA), carbohydrates, and/or viruses, such as oncolytic viruses. The cells may express or in any event produce one or more biomolecules, including at least a recombinant therapeutic and/or diagnostic product. Examples of biomolecules include at least antibody molecules of any type (e.g., monoclonal antibodies, bispecific antibodies), antibody mimetics (including at least DARPins, affibodies, adnectins, or IgNARs), fusion proteins (e.g., Fc fusion proteins, chimeric cytokines, chimeric antigen receptors), other recombinant proteins (e.g., glycosylated proteins, enzymes, hormones), viral therapeutics (e.g., anti-cancer oncolytic viruses and/or viral vectors for gene therapy and viral immunotherapy), cell therapeutics (e.g., pluripotent stem cells, mesenchymal stem cells and/or adult stem cells, and these may or may not be engineered themselves), vaccines or lipid-encapsulated particles (e.g., exosomes, virus-like particles), RNA (such as e.g. siRNA) or DNA (such as e.g. plasmid DNA), antibiotics and/or amino acids.

In certain embodiments, the amount of biomolecule produced by the culture is at least 1 gram, at least 10 grams, at least 100 grams, 500 grams, 1000 grams, 2000 grams, 3000 grams, or more of a molecule. In some embodiments, a manufacturing-scale bioreactor culture produces or is 5 capable of producing 1 gram to 10 grams, 1 gram to 100 grams, 1 gram to 500 grams, 10 gram to 1000 grams, 10 grams to 2000 grams, 100 grams to 1000 grams, 500 grams to 5000 grams, or more of a molecule.

The cells for the cell culture may originate from a cell line, in some embodiments. Examples of such cells, cell lines or cell strains include e.g. mouse myeloma (NSO)-cell lines, Chinese hamster ovary (CHO)-cell lines, Jurkat, NIH3T3, COS, K562, HEK-293, VERO, PER.C6, HeLA, EB1, EB2, EB3, oncolytic and/or hybridoma-cell lines.

In one embodiment, the cells of the culture may comprise stem cells that may or may not be, for example, pluripotent stem cells, including embryonic stem cells (ESCs), adult stem cells, induced pluripotent stem cells (iPSCs), tissue specific stem cells (e.g., hematopoietic stem cells) mesenchymal stem cells (MSCs) or a mixture thereof.

In one embodiment, the cells may be immune cells of any kind, including T cells, natural killer cells, natural killer T cells, B cells, dendritic cells, tumor infiltrating lymphocytes, monocytes, megakaryocytes, a combination thereof, and so forth. The cells of the cell culture may comprise a differentiated form of any of the cells described herein. In one embodiment, the cell is cell derived from any primary cell in culture.

In some embodiments, a polypeptide (such as an antibody) expressed by the cells of the present disclosure may bind to, or interact with, any protein, including, without limitation, cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL1 0, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-0), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.k.

In some embodiments, a polypeptide (such as an antibody) expressed by the cells of the present disclosure may bind to, or interact with, a chemokine, chemokine receptor, or a chemokine-related protein selected from the group consisting of CCLI (1-309), CCL2 (MCP-1/MCAF), CCL3 (MIP-Iα), CCL4 (MIP-Iβ), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL. In some embodiments, the polypeptide expressed by the host cells of the present disclosure may bind to, or interact with, 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AVMR2; amyloid beta; ANGPTL; ANGPT2; ANGPTL3; ANGPTL4; ANPEP; APC; APOC1; AR; ASLG659; ASPHDI (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP16); CCL16 (HCC-4); CCL17 (TARC); CCL18 (PARC); CCL19 (MIP-30); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79α, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsinB); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-α; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL2Ra; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67, RIG-E, SCA-2, TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAMI; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B); SERPINA1; SERPINA3; SERPINB5 (maspin); SERPINE1(PAI-1); SERPDMF1; SHBG; SLA2; SLC2A2; SLC33A1; SLC43A1; SLIT2; SPP1; SPRR1B (Spr1); ST6GAL1; STAB1; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-1b); XCRI (GPR5/CCXCRI); YY1; and/or ZFPM2.

In certain embodiments, target molecules for antibodies (or bispecific antibodies) produced according to the methods disclosed herein include CD proteins such as CD3, CD4, CDS, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD790, IGb (immunoglobulin-associated beta), B29); CD200 members of the ErbB receptor family such as the EGF receptor, HER2, HER3, or HER4 receptor; cell adhesion molecules such as LFA-1, Maci, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc. In certain embodiments, the methods provided herein can be used to produce an antibody (or a multispecific antibody, such as a bispecific antibody) that specifically binds to complement protein C5 (e.g., an anti-C5 agonist antibody that specifically binds to human C5).

### IX. Recitation of Various Embodiments of the Present Disclosure

Embodiment 1: A method for predicting cell viability of a cell culture in a bioreactor during a biomolecule manufacturing process, the method comprising: receiving at least three manufacturing process parameters selected from one or both of a first set of manufacturing process parameters or a second set of manufacturing process parameters measured from the cell culture during the biomolecule manufacturing process; analyzing the at least three manufacturing process parameters using a trained machine learning model to generate an indicator of cell viability of the cell culture, wherein: any of the first set of manufacturing process parameters has an order of effect on the indicator of cell viability of the cell culture that is higher than that of any of the second set of manufacturing process parameters; the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; and generating the indicator of cell viability of the cell culture based on the analyzing.

Embodiment 2: The method of embodiment 1, wherein the trained machine learning model is a neural network.

Embodiment 3: The method of embodiment 1, wherein the trained machine learning model is a decision-tree based machine learning model.

Embodiment 4: The method of any of embodiments 1-3, wherein the trained machine learning model is trained with a manufacturing process training record including the one or both of the first set of manufacturing process parameters or the second set of manufacturing process parameters and the indicator of cell viability.

Embodiment 5: The method of any of embodiments 1-4, wherein at least one manufacturing process parameter of the one or both of the first set of manufacturing process parameters or the second set of manufacturing process parameters is measured by a sensor operationally connected to the bioreactor.

Embodiment 6: The method of embodiment 5, wherein the at least one manufacturing process parameter is the pH of the cell culture, the temperature of the cell culture, the amount of dissolved oxygen in the cell culture, or a total volume of the cell culture, and the sensor is a temperature probe, a dissolved oxygen probe, a pH probe, or a scale configured to weigh the cell culture, respectively, disposed within the bioreactor.

Embodiment 7: The method of any of embodiments 1-4, wherein at least one manufacturing process parameter of the one or both of the first set of manufacturing process parameters or the second set of manufacturing process parameters is an output of a controller operationally connected to the bioreactor.

Embodiment 8: The method of embodiment 7, wherein the at least one manufacturing process parameter is an amount of air sparged into the cell culture, an amount of carbon dioxide sparged into the cell culture, or an amount of oxygen sparged into the cell culture, and the controller is an air flow controller configured to control flow of the air sparged into the cell culture, the carbon dioxide sparged into the cell culture, or the oxygen sparged into the cell culture, respectively.

Embodiment 9: The method of any of embodiments 1-8, wherein the biomolecules include a monoclonal antibody, a complex antibody, an antibody fragment, a virus or a viral particle a biopharmaceutical, a cytokine, a fusion protein, a growth factor, an immunogenic composition, a vaccine, a lipid, a carbohydrate, and/or a nucleic acid and/or wherein the cells in the cell culture produce a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid.

Embodiment 10: The method of any of embodiments 1-9, wherein: the first set of manufacturing process parameters further includes an amount of oxygen sparged into the cell culture; the first set of manufacturing process parameters listed in order of effect on the indicator of cell viability is shown in Table 1; and Table 1=

| Serial No. | Manufacturing process parameter |
|---|---|
| 1 | the time elapsed since the |
| | initiation of the biomolecule |
| | manufacturing process |
| 2 | the amount of oxygen sparged into |
| | the cell culture |
| 3 | the total volume of the cell culture |
| 4 | the total base added into the cell |
| | culture during the biomolecule |
| | manufacturing process |

Embodiment 11: The method of embodiment 10, wherein the second set of manufacturing process parameters further includes an amount of carbon dioxide sparged into the cell culture; the second set of manufacturing process parameters listed in order of effect on the indicator of cell viability is shown in Table 2; and Table 2=

| Serial No. | Manufacturing process parameter |
|---|---|
| 1 | the amount of carbon dioxide |
| | sparged into the cell culture |
| 2 | the amount of dissolved oxygen in |
| | the cell culture |
| 3 | the pH of the cell culture |
| 4 | the amount of air sparged into the |
| | cell culture |
| 5 | the temperature of the cell culture |

Embodiment 12: The method of any of embodiments 1-9, wherein: the first set of manufacturing process parameters listed in order of effect on the indicator of cell viability is shown in Table 3; and Table 3=

| Serial No. | Manufacturing process parameter |
|---|---|
| 1 | the time elapsed since the |
| | initiation of the biomolecule |
| | manufacturing process |
| 2 | the total base added into the cell |
| | culture during the biomolecule |
| | manufacturing process |
| 3 | the total volume of the cell culture |

Embodiment 13: The method of embodiment 12, wherein: the second set of manufacturing process parameters listed in order of effect on the indicator of cell viability is shown in Table 4; and Table 4=

| Serial No. | Manufacturing process parameter |
|---|---|
| 1 | the amount of oxygen sparged into |
| | the cell culture |
| 2 | the amount of carbon dioxide |
| | sparged into the cell culture |
| 3 | the temperature of the cell culture |
| 4 | the amount of air sparged into the |
| | cell culture |
| 5 | the pH of the cell culture |
| 6 | the amount of dissolved oxygen in |
| | the cell culture |

Embodiment 14: The method of embodiment 12, wherein: the second set of manufacturing process parameters listed in order of effect on the indicator of cell viability is shown in Table 5; and Table 5=

| Serial No. | Manufacturing process parameter |
|---|---|
| 1 | the amount of air sparged into the |
| | cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of carbon dioxide |
| | sparged into the cell culture |

Embodiment 15: The method of embodiment 12, wherein: the second set of manufacturing process parameters listed in order of effect on the indicator of cell viability is shown in Table 6; and Table 6=

| Serial No. | Manufacturing process parameter |
|---|---|
| 1 | the pH of the cell culture |
| 2 | the amount of carbon dioxide |
| | sparged into the cell culture |
| 3 | the temperature of the cell culture |
| 4 | the amount of oxygen sparged into |
| | the cell culture |
| 5 | the amount of air sparged into the |
| | cell culture |
| 6 | the amount of dissolved oxygen in |
| | the cell culture |

Embodiment 16: The method of any of embodiments 1-9, wherein: the first set of manufacturing process parameters further includes an amount of carbon dioxide sparged into the cell culture; the first set of manufacturing process parameters listed in order of effect on the indicator of cell viability is shown in Table 7; and Table 7=

| Serial No. | Manufacturing process parameter |
|---|---|
| 1 | the time elapsed since the |
| | initiation of the biomolecule |
| | manufacturing process |
| 2 | the amount of carbon dioxide |
| | sparged into the cell culture |
| 3 | the total base added into the cell |
| | culture during the biomolecule |
| | manufacturing process |
| 4 | the total volume of the cell culture |

Embodiment 17: The method of embodiment 16, wherein: the second set of manufacturing process parameters further includes an amount of oxygen sparged into the cell culture; the second set of manufacturing process parameters listed in order of effect on the indicator of cell viability is shown in Table 8; and Table 8=

| Serial No. | Manufacturing process parameter |
|---|---|
| 1 | the amount of air sparged into the |
| | cell culture |
| 2 | the amount of dissolved oxygen in |
| | the cell culture |
| 3 | the temperature of the cell culture |
| 4 | the pH of the cell culture |
| 5 | the amount of oxygen sparged into |
| | the cell culture |

Embodiment 18: The method of embodiment 16, wherein: the second set of manufacturing process parameters further includes an amount of oxygen sparged into the cell culture; the second set of manufacturing process parameters listed in order of effect on the indicator of cell viability is shown in Table 9; and Table 9=

| Serial No. | Manufacturing process parameter |
|---|---|
| 1 | the temperature of the cell culture |
| 2 | the amount of oxygen sparged into |
| 3 | the pH of the cell culture |
| 4 | the amount of air sparged into the |
| | cell culture |
| 5 | the cell culture the amount of |
| | dissolved oxygen in the cell |
| | culture |

Embodiment 19: The method of any of embodiments 1-9, wherein: the first set of manufacturing process parameters listed in order of effect on the indicator of cell viability is shown in Table 10; and Table 10=

| Serial No. | Manufacturing process parameter |
|---|---|
| 1 | the time elapsed since the |
| | initiation of the biomolecule |
| | manufacturing process |
| 2 | the total volume of the cell culture |
| 3 | the total base added into the cell |
| | culture during the biomolecule |
| | manufacturing process |

Embodiment 20: The method of embodiment 19, wherein: the second set of manufacturing process parameters listed in order of effect on the indicator of cell viability is shown in Table 11; and Table 11=

| Serial No. | Manufacturing process parameter |
|---|---|
| 1 | the amount of carbon dioxide |
| | sparged into the cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of air sparged into the |
| | cell culture |

Embodiment 21. A system for predicting viability of cells producing biomolecules during a process for manufacturing the biomolecules in a cell culture in a bioreactor, the system comprising: a non-transitory memory storing instructions; and a processor coupled to the non-transitory memory and configured to read the instructions from the non-transitory memory to cause the system to perform any of the methods of embodiments 1-20.

Embodiment 22. A non-transitory computer-readable medium (CRM) having stored thereon computer-readable instructions executable to cause performance of operations for predicting viability of cells producing biomolecules during a process for manufacturing the biomolecules in a cell culture in a bioreactor, the operations comprising any of the methods of embodiments 1-20.

Embodiment 23. A method for predicting cell viability of a cell culture in a bioreactor during a biomolecule manufacturing process, the method comprising: receiving at least three manufacturing process parameters collectively selected from a first set of manufacturing process parameters and a second set of manufacturing process parameters measured from the cell culture during the biomolecule manufacturing process; analyzing the at least three manufacturing process parameters using a trained machine learning model to generate an indicator of cell viability of the cell culture, wherein: optionally, any of the first set of manufacturing process parameters has an order of effect on the indicator of cell viability of the cell culture that is higher than that of any of the second set of manufacturing process parameters; the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; and generating the indicator of cell viability of the cell culture based on the analyzing.

Embodiment 24. A method for predicting cell viability of a cell culture in a bioreactor during a biomolecule manufacturing process, the method comprising: receiving at least three manufacturing process parameters selected from one or both of a first set of manufacturing process parameters or a second set of manufacturing process parameters measured from the cell culture during the biomolecule manufacturing process; analyzing the at least three manufacturing process parameters using a trained machine learning model to generate an indicator of cell viability of the cell culture, wherein: any of the first set of manufacturing process parameters has an order of effect on the indicator of cell viability of the cell culture that is higher than that of any of the second set of manufacturing process parameters; the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; and generating the indicator of cell viability of the cell culture based on the analyzing.

Embodiment 25. A method for predicting cell viability of a cell culture in a bioreactor during a biomolecule manufacturing process, the method comprising: receiving at least three manufacturing process parameters selected from one or both of a first set of manufacturing process parameters or a second set of manufacturing process parameters measured from the cell culture during the biomolecule manufacturing process; analyzing the at least three manufacturing process parameters using a trained machine learning model to generate an indicator of cell viability of the cell culture, wherein: the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; and generating the indicator of cell viability of the cell culture based on the analyzing.

Embodiment 26: A method for predicting cell viability of a cell culture (optionally in a bioreactor and/or also optionally wherein the cells produce biomolecules), comprising: receiving data corresponding to at least three manufacturing process parameters selected from one or both of a first set of manufacturing process parameters or a second set of manufacturing process parameters, wherein: the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; inputting the data into a trained machine learning model; and analyzing the data using the trained machine learning model to generate an indicator of cell viability of the cell culture.

Embodiment 27: The method of embodiment 26, wherein the biomolecule is a biopharmaceutical, antibody, cytokine, fusion protein, growth factors, immunogenic composition, vaccine, lipid, carbohydrate; or nucleic acid.

Embodiment 28: The method of embodiment 26, wherein any of the first set of manufacturing process parameters has an order of effect on the indicator of cell viability of the cell culture that is higher than that of any of the second set of manufacturing process parameters.

Embodiment 29: The method of embodiment 26, wherein the trained machine learning model is a neural network.

Embodiment 30: The method of embodiment 26, wherein the trained machine learning model is a decision-tree based machine learning model.

Embodiment 31: The method of embodiment 26, wherein the cells are eukaryotic cells.

Embodiment 32: A computer-implemented method for predicting cell viability of cells in a cell culture, the method comprising: determining at least three manufacturing process parameters selected from one or both of a first set of manufacturing process parameters or a second set of manufacturing process parameters, wherein: the first set of process parameters includes time elapsed since an initiation of the biomolecule manufacturing process, total base added into the cell culture during the biomolecule manufacturing process, and a total volume of the cell culture in the bioreactor; and the second set of process parameters includes an amount of air sparged into the cell culture, an amount of dissolved oxygen in the cell culture, a pH of the cell culture, and a temperature of the cell culture; training a machine learning model using a training set comprising the at least three manufacturing process parameters to produce an output; and generating a predicted cell viability of the cells in the cell culture based on the output. Embodiment 33: The method of embodiment 32, wherein any of the first set of manufacturing process parameters has an order of effect on the indicator of cell viability of the cell culture that is higher than that of any of the second set of manufacturing process parameters.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A computer-implemented method for predicting cell viability of a cell culture in a bioreactor during a biomolecule manufacturing process, the method comprising:
receiving at least three manufacturing process parameters selected from one or both of a first set of manufacturing process parameters or a second set of manufacturing process parameters measured from the cell culture during the biomolecule manufacturing process;
generating the indicator of cell viability of the cell culture by providing at least three manufacturing process parameters as input to a machine learning model that has been trained to generate an indicator of cell viability of the cell culture using training data comprising, for each of a plurality of cell cultures in a biomolecule manufacturing process, values of the at least three manufacturing process parameters and corresponding measured values of the indicator of cell viability, wherein:
the first set of process parameters includes time elapsed since an initiation of the biomolecule manufacturing process, total base added into the cell culture during the biomolecule manufacturing process, and a total volume of the cell culture in the bioreactor; and
the second set of process parameters includes an amount of air sparged into the cell culture, an amount of dissolved oxygen in the cell culture, a pH of the cell culture, and a temperature of the cell culture.

2. The method of claim 1, wherein any of the first set of manufacturing process parameters has an order of effect on the predicted indicator of cell viability of the cell culture that is higher than that of any of the second set of manufacturing process parameters.

3. The method of claim 1, wherein the trained machine learning model is a neural network or a decision-tree based machine learning model.

4. (canceled)

5. The method of claim 1, wherein the trained machine learning model has been trained with a manufacturing process training record including the one or both of the first set of manufacturing process parameters or the second set of manufacturing process parameters and the indicator of cell viability.

6. The method of claim 1, wherein the at least three manufacturing process parameters comprise one or more manufacturing process parameters that are measured by a sensor operationally connected to the bioreactor.

7. The method of claim 6, wherein the one or more manufacturing process parameters are selected from: the pH of the cell culture, the temperature of the cell culture, the amount of dissolved oxygen in the cell culture, and a total volume of the cell culture, and/or the one or more manufacturing process parameters are each measured by a sensor selected from: a temperature probe, a dissolved oxygen probe, a pH probe, a Raman probe, a fluorescent probe, a liquid level imaging sensor, and a scale configured to weigh the cell culture, disposed within the bioreactor.

8. The method of claim 1, wherein the three manufacturing process parameters comprise one or more manufacturing process parameters that are obtained as an output of a controller operationally connected to the bioreactor.

9. The method of claim 8, wherein the one or more manufacturing process parameters are selected from: an amount of air sparged into the cell culture, an amount of carbon dioxide sparged into the cell culture, and an amount of oxygen sparged into the cell culture, Raman spectral values, fluorescence values, and/or, the one or more manufacturing process parameters are each obtained as the output of an air flow controller configured to control flow of one or more of: the air sparged into the cell culture, the carbon dioxide sparged into the cell culture, and the oxygen sparged into the cell culture.

10. (canceled)

11. The method of claim 1, wherein:
the first set of manufacturing process parameters further includes an amount of oxygen sparged into the cell culture;
the first set of manufacturing process parameters listed in order of effect on the indicator of cell viability is shown in Table 1; and
Table 1=

| Serial No. | Manufacturing process parameter |
|---|---|
| 1 | the time elapsed since the |
| | initiation of the biomolecule |
| | manufacturing process |
| 2 | the amount of oxygen sparged into |
| | the cell culture |
| 3 | the total volume of the cell culture |
| 4 | the total base added into the cell |
| | culture during the biomolecule |
| | manufacturing process |

12. The method of claim 11, wherein:
the second set of manufacturing process parameters further includes an amount of carbon dioxide sparged into the cell culture;
the second set of manufacturing process parameters listed in order of effect on the indicator of cell viability is shown in Table 2; and
Table 2=

| Serial No. | Manufacturing process parameter |
|---|---|
| 1 | the amount of carbon dioxide |
| | sparged into the cell culture |
| 2 | the amount of dissolved oxygen in |
| | the cell culture |
| 3 | the pH of the cell culture |
| 4 | the amount of air sparged into the |
| | cell culture |
| 5 | the temperature of the cell culture |

13. The method of claim 1, wherein:
the first set of manufacturing process parameters listed in order of effect on the indicator of cell viability is shown in Table 3; and
Table 3=

| Serial No. | Manufacturing process parameter |
|---|---|
| 1 | the time elapsed since the |
| | initiation of the biomolecule |
| | manufacturing process |
| 2 | the total base added into the cell |
| | culture during the biomolecule |
| | manufacturing process |
| 3 | the total volume of the cell culture |

14. The method of claim 13, wherein:
the second set of manufacturing process parameters listed in order of effect on the indicator of cell viability is shown in Table 4; and
Table 4=

| Serial No. | Manufacturing process parameter |
|---|---|
| 1 | the amount of oxygen sparged into |
| | the cell culture |
| 2 | the amount of carbon dioxide |
| | sparged into the cell culture |
| 3 | the temperature of the cell culture |
| 4 | the amount of air sparged into the |
| | cell culture |
| 5 | the pH of the cell culture |
| 6 | the amount of dissolved oxygen in |
| | the cell culture |

15. The method of claim 13, wherein:
the second set of manufacturing process parameters listed in order of effect on the indicator of cell viability is shown in Table 5; and
Table 5=

| Serial No. | Manufacturing process parameter |
|---|---|
| 1 | the amount of air sparged into the |
| | cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of carbon dioxide |
| | sparged into the cell culture |

16. The method of claim 13, wherein:
the second set of manufacturing process parameters listed in order of effect on the indicator of cell viability is shown in Table 6; and
Table 6=

| Serial No. | Manufacturing process parameter |
|---|---|
| 1 | the pH of the cell culture |
| 2 | the amount of carbon dioxide |
| | sparged into the cell culture |
| 3 | the temperature of the cell culture |
| 4 | the amount of oxygen sparged into |
| | the cell culture |
| 5 | the amount of air sparged into the |
| | cell culture |
| 6 | the amount of dissolved oxygen in |
| | the cell culture |

17. The method of claim 1, wherein:
the first set of manufacturing process parameters further includes an amount of carbon dioxide sparged into the cell culture;
the first set of manufacturing process parameters listed in order of effect on the indicator of cell viability is shown in Table 7; and
Table 7=

| Serial No. | Manufacturing process parameter |
|---|---|
| 1 | the time elapsed since the |
| | initiation of the biomolecule |
| | manufacturing process |
| 2 | the amount of carbon dioxide |
| | sparged into the cell culture |
| 3 | the total base added into the cell |
| | culture during the biomolecule |
| | manufacturing process |
| 4 | the total volume of the cell culture |

18. The method of claim 15, wherein:
the second set of manufacturing process parameters further includes an amount of oxygen sparged into the cell culture;
the second set of manufacturing process parameters listed in order of effect on the indicator of cell viability is shown in Table 8; and
Table 8=

| Serial No. | Manufacturing process parameter |
|---|---|
| 1 | the amount of air sparged into the |
| | cell culture |
| 2 | the amount of dissolved oxygen in |
| | the cell culture |
| 3 | the temperature of the cell culture |
| 4 | the pH of the cell culture |
| 5 | the amount of oxygen sparged into |
| | the cell culture |

19. The method of claim 17, wherein:
the second set of manufacturing process parameters further includes an amount of oxygen sparged into the cell culture;
the second set of manufacturing process parameters listed in order of effect on the indicator of cell viability is shown in Table 9; and
Table 9=

| Serial No. | Manufacturing process parameter |
|---|---|
| 1 | the temperature of the cell culture |
| 2 | the amount of oxygen sparged into |
| 3 | the pH of the cell culture |
| 4 | the amount of air sparged into the |
| | cell culture |
| 5 | the cell culture the amount of |
| | dissolved oxygen in the cell |
| | culture |

20. The method of claim 1, wherein:
the first set of manufacturing process parameters listed in order of effect on the indicator of cell viability is shown in Table 10; and
Table 10=

| Serial No. | Manufacturing process parameter |
|---|---|
| 1 | the time elapsed since the |
| | initiation of the biomolecule |
| | manufacturing process |
| 2 | the total volume of the cell culture |
| 3 | the total base added into the cell |
| | culture during the biomolecule |
| | manufacturing process |

21. The method of claim 20, wherein:
the second set of manufacturing process parameters listed in order of effect on the indicator of cell viability is shown in Table 11; and
Table 11=

| Serial No. | Manufacturing process parameter |
|---|---|
| 1 | the amount of carbon dioxide |
| | sparged into the cell culture |
| 2 | the pH of the cell culture |
| 3 | the amount of oxygen sparged into |
| | the cell culture |
| 4 | the amount of dissolved oxygen in |
| | the cell culture |
| 5 | the temperature of the cell culture |
| 6 | the amount of air sparged into the |
| | cell culture |

22. The method of claim 1, wherein the trained machine learning model comprises parameters that assign higher importance to the manufacturing process parameters selected from the first set than to the manufacturing process parameters selected from the second set, optionally wherein the trained machine learning model comprises parameters that assign respective importance to each of the manufacturing process parameters, wherein:
(i) the at least three manufacturing process parameters are selected from or include all of the parameters of Tables 1 and 2, and the order of importance of the manufacturing process parameters are provided in Tables 1 and 2;
(ii) the at least three manufacturing process parameters are selected from or include all of the parameters of Tables 3 and 4, and the order of importance of the manufacturing process parameters are provided in Tables 3 and 4;
(iii) the at least three manufacturing process parameters are selected from or include all of the parameters of Tables 5 and 6, and the order of importance of the manufacturing process parameters are provided in Tables 5 and 6;
(iv) the at least three manufacturing process parameters are selected from or include all of the parameters of Tables 7 and 8, and the order of importance of the manufacturing process parameters are provided in Tables 7 and 8;
(v) the at least three manufacturing process parameters are selected from or include all of the parameters of Tables 7 and 9, and the order of importance of the manufacturing process parameters are provided in Tables 7 and 9;
(vi) the at least three manufacturing process parameters are selected from or include all of the parameters of Tables 10 and 11, and the order of importance of the manufacturing process parameters are provided in Tables 10 and 11.

23. (canceled)

24. A system for predicting cell viability of a cell culture in a bioreactor during a biomolecule manufacturing process, the system comprising:
a non-transitory memory storing instructions; and
a processor coupled to the non-transitory memory and configured to read the instructions from the non-transitory memory to cause the system to perform operations comprising:
receiving at least three manufacturing process parameters selected from one or both of a first set of manufacturing process parameters or a second set of manufacturing process parameters measured from the cell culture during the biomolecule manufacturing process;
analyzing the at least three manufacturing process parameters using a trained machine learning model to generate an indicator of cell viability of the cell culture, wherein:
any of the first set of manufacturing process parameters has an order of effect on the indicator of cell viability of the cell culture that is higher than that of any of the second set of manufacturing process parameters;
the first set of process parameters includes time elapsed since an initiation of the biomolecule manufacturing process, total base added into the cell culture during the biomolecule manufacturing process, and a total volume of the cell culture in the bioreactor; and
the second set of process parameters includes an amount of air sparged into the cell culture, an amount of dissolved oxygen in the cell culture, a pH of the cell culture, and a temperature of the cell culture; and
generating the indicator of cell viability of the cell culture based on the analyzing.

25. A non-transitory computer-readable medium (CRM) having stored thereon computer-readable instructions executable to cause performance of operations A system for predicting cell viability of a cell culture in a bioreactor during a biomolecule manufacturing process, the operations comprising:
receiving at least three manufacturing process parameters selected from one or both of a first set of manufacturing process parameters or a second set of manufacturing process parameters measured from the cell culture during the biomolecule manufacturing process;
analyzing the at least three manufacturing process parameters using a trained machine learning model to generate an indicator of cell viability of the cell culture, wherein:
any of the first set of manufacturing process parameters has an order of effect on the indicator of cell viability of the cell culture that is higher than that of any of the second set of manufacturing process parameters;
the first set of process parameters includes time elapsed since an initiation of the biomolecule manufacturing process, total base added into the cell culture during the biomolecule manufacturing process, and a total volume of the cell culture in the bioreactor; and
the second set of process parameters includes an amount of air sparged into the cell culture, an amount of dissolved oxygen in the cell culture, a pH of the cell culture, and a temperature of the cell culture; and
generating the indicator of cell viability of the cell culture based on the analyzing.

26. The method of any preceding claim, wherein the at least three manufacturing process parameters do not include any manufacturing process parameter that is obtained by analyzing cells sampled from the cell culture and/or that is measured by analyzing a sample of cell culture obtained from the bioreactor.

27. (canceled)
